(19) United States
(12) Patent Application Publication
(10) Pub. No.: US 2010/0256105 A1
(43) Pub. Date: Oct. 7, 2010

(54) NOVEL COMPOUNDS

(75) Inventors: Frank Burkamp, Sodertalje (SE); Peter Hansen, Lund (SE)

Correspondence Address: ASTRAZENECA R&D BOSTON, 35 GATEHOUSE DRIVE, WALTHAM, MA, 02451-1215, US

(73) Assignee: AstraZeneca R&D

(21) Appl. No.: 12/749,919

(22) Filed: Mar. 30, 2010

Related U.S. Application Data
(60) Provisional application No. 61/166,323, filed on Apr. 3, 2009.

(51) Int. Cl. A61K 31/58; C07J 71/00; A61P 11/06; A61P 11/02

(52) U.S. Cl. 514/176; 540/53

(57) ABSTRACT

The present invention provides compounds of formula (I)

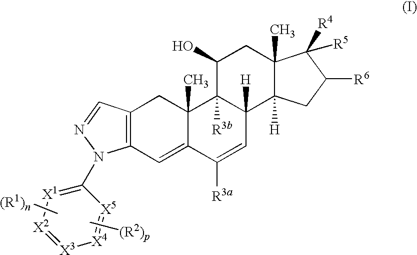

wherein n, p, R¹, R², X¹, X², X³, X⁴, X⁵, R^{3a}, R^{3b}, R⁴, R⁵ and R⁶ are as defined in the specification, a process for their preparation, pharmaceutical compositions containing them and their use in therapy.

This application claims the benefit of U.S. Provisional application No. 61/166,323, filed Apr. 3, 2009, the disclosure of which is incorporated by reference herein in its entirety.

The present invention relates to compounds having glucocorticosteroid receptor agonist activity, processes for their preparation, pharmaceutical compositions containing them and their therapeutic use, particularly for the treatment of inflammatory and allergic conditions.

Glucocorticosteroids (GCs) that have anti-inflammatory properties are known and are widely used for the treatment of diseases such as inflammatory arthritides (e.g. rheumatoid arthritis, ankylosing spondylitis and psoriatic arthropathy), other rheumatoid diseases such as systemic lupus erythematosis, scleroderma, vascutitides including temporal arteritis and polyarteritis nodosa, inflammatory bowel disease such as Crohns disease and ulcerative colitis, lung diseases such as asthma and chronic obstructive airways disease, as well as many other conditions such as polymyalgia rheumatica. GCs have also been used very extensively for their immunosuppressive properties in the prevention and treatment of transplant rejection. Finally GCs have been used for their anti-tumour effects in a number of malignancies.

GCs act via specific glucocorticoid receptors (GR) that are members of the nuclear receptor superfamily. Ligand binding promotes receptor dimerisation, DNA binding, and transcriptional activation. This mechanism of GC action is well defined in vitro and is critical for regulation of the hypothalamic-pituitary-adrenal axis, gluconeogenesis as well as transcription of anti-inflammatory genes such as mitogen-activated protein kinase phosphatase-1 (MKP-1) and secretory leukocyte protease inhibitor (SLPI) in vivo. Ligand-bound receptor is also able to suppress gene transcription in a dimerisation-independent manner by interfering with the activity of transcription factors, such as AP-1 and NFkB, which are critically involved in the inflammatory reaction.

After ligand binding, the GR translocates from the cytoplasm of the cell to the nucleus and binds to glucocorticoid response elements in regulator regions of target genes. The activated GR then recruits co-factors, including the glucocorticoid receptor interacting protein 1 (GRIP-1) and steroid receptor co-activator 1 (SRC1). These accessory proteins bind to the receptor and link the GR with the general transcription machinery to drive transcription of target genes.

Glucocorticoid effects on transcription may be mediated by both the direct binding of activated GR to target DNA, homodimerisation and recruitment of co-activators (known as “transactivation”) but also by GR interfering with other transcription factor function, including AP-1 and NFkB, by complexing with these other transcription factors and preventing them from binding to their target genes leading to repression of the genes normally upregulated by AP-1 or NFkB (known as “transrepression”). These two modes of receptor activity are dissociable and negative effects on NFkB activity can be retained in the absence of transactivation. It appears that transrepression is largely responsible for mediating the therapeutically desirable anti-inflammatory activity of the GR. Interestingly, the IC₅₀ for inhibition of AP-1 or NFkB (0.04 nM) is lower than the EC₅₀ for activation of target genes (5 nM) and yet high doses of GCs are frequently required to treat patients with inflammatory disease. One explanation is that cytokines expressed at the site of inflammation may induce relative glucocorticoid resistance, for instance by activating AP-1 or NFkB. This is of importance as many pro-inflammatory cytokines signal by activation of NFkB and a major anti-inflammatory action of GCs is thought to be mediated by opposing NFkB action.

Published Japanese Patent Application No. 60067495 describes certain pregnenopyrazoles as anti-inflammatory agents.

In accordance with the present invention, there is provided a compound of formula

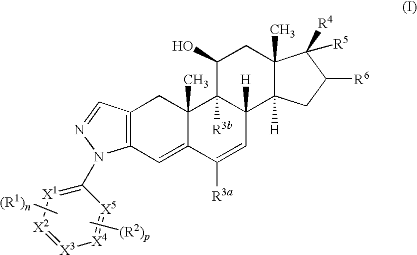

wherein
X¹, X², X³, X⁴ and X⁵ each independently represent CH or a nitrogen atom, provided that no more than two of X¹, X², X³, X⁴ and X⁵ may simultaneously represent a nitrogen atom;
n and p each independently represent 0 or 1;
R¹ represents a halogen atom or a methyl or a methoxy group;

R² represents a halogen atom, —C(O)OCH₃, —C(O)—S—CH₂CN, —C(O)—S—CH₃, —C(O)-heterocyclyl, —SO₂CH₃, a C₂-C₆ alkenyl group, or a methyl group optionally substituted by halogen, hydroxyl, methoxy, —OCH₂CH═CH₂ or —NR⁷R⁸;
R^{3a} represents a hydrogen atom or methyl group and R^{3b} represents a hydrogen or fluorine atom;
R⁴ represents —C(O)—S—C(O)N(CH₃)₂, —C(O)CH₂Cl, —C(O)—Y—CH(R¹¹)—R⁹ or —C(O)—CH(R¹¹)—Y—R⁹;
R⁵ represents —OCH₂SCH₃, —O—C(O)—R¹⁰, —O—C(O)—NH—R¹⁰, —O—C(O)—O—R¹⁰ or —O—C(O)—S—R¹⁰;
R⁶ represents a hydrogen or a halogen atom or a hydroxyl or methyl group;
R⁷ and R⁸ each independently represent a hydrogen atom, or a C₁-C₃ alkyl or a C₁-C₃ hydroxyalkyl group, or
R⁷ and R⁸ together with the nitrogen atom to which they are attached form a 3- to 8-membered saturated or partially saturated heterocyclic ring optionally containing a further ring heterogroup selected from nitrogen, S(O)ₘ and oxygen, the heterocyclic ring being optionally substituted by at least one substituent selected from hydroxyl, C₁-C₃ alkyl and C₁-C₃ hydroxyalkyl;
m is 0, 1 or 2;
Y represents an oxygen or sulphur atom or a group>NH;
R⁹ represents hydrogen, halogen, cyano, —S—CN, —C(O)N(R¹²)₂, C₁-C₆ alkoxycarbonyl, C₁-C₆ alkylcarbonyl (optionally substituted by —OC(O)CH₃), C₁-C₆ alkylcarbonyloxy, C₁-C₆ alkoxy, C₁-C₆ alkylthio, —C(O)—S—C₁-C₆ alkyl, —C(═CH₂)—O—CH₂OCH₃, C₁-C₆ alkyl, C₂-C₆ alkenyl, C₂-C₆ alkynyl or C₃-C₇ cycloalkyl, the latter four groups being optionally substituted by one or more substituents independently selected from halogen, hydroxyl, cyano, hydroxymethyl, C₁-C₄ alkoxy and C₁-C₄ alkylcarbonyloxy;
R¹⁰ represents C₁-C₆ alkyl (optionally substituted by halogen, C₁-C₄ alkoxy, C₁-C₄ alkylcarbonyloxy or C₃-C₇ cycloalkyl) or a 3- to 10-membered saturated or unsaturated carbocyclic or heterocyclic ring system which ring system may be optionally substituted by at least one substituent selected from halogen, carboxyl, hydroxyl, oxo, nitro, cyano, mercapto, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylthio, C₁-C₆ alkylsulphinyl, C₁-C₆ alkylsulphonyl, C₁-C₆ alkylcarbonyl, C₁-C₆ alkylcarbonyloxy, C₁-C₆ alkoxycarbonyl, amino (—NH₂), carboxamido (—CONH₂), (mono)C₁-C₆ alkylamino, (di)C₁-C₆ alkylamino and phenyl;
R¹¹ represents a hydrogen atom or a methyl group; and
each R¹² independently represents a hydrogen atom or a methyl group;
   or a pharmaceutically acceptable salt thereof.

In the context of the present specification, unless otherwise stated, an alkyl, alkenyl or alkynyl substituent group or an alkyl, alkenyl or alkynyl moiety in a substituent group may be linear or branched. Examples of C₁-C₆ alkyl groups/moieties include methyl, ethyl, propyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, tert-butyl, n-pentyl, isopentyl, neopentyl and n-hexyl. Examples of C₂-C₆ alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1-hexadienyl. Examples of C₂-C₆ alkynyl groups/moieties include ethynyl, propynyl, 1-butynyl, 2-butynyl, 1-pentynyl and 1-hexynyl.

An alkylene, alkenylene or alkynylene linking group may be cyclic, linear or branched and may contain, for example, up to a total of eight carbon atoms. Examples of C₁-C₆ alkylene linking groups include methylene, ethylene, n-propylene, n-butylene, n-pentylene, n-hexylene, 1-methylethylene, 2-methylethylene, 1,2-dimethylethylene, 1-ethylethylene, 2-ethylethylene, 1-, 2- or 3-methylpropylene and 1-, 2- or 3-ethylpropylene; C₂-C₆ alkenylene linking groups containing one or more carbon-carbon double bonds include vinylidene, ethenylene (vinylene), propenylene, methylethenylene, 1-propenylidene, 2-propenylidene, 3-methylpropenylene, 3-ethylpropenylene, 1,3-dimethylpropenylene, 2,3-dimethylpropenylene, 3,3-dimethylpropenylene, 3-ethyl-1-methylpropenylene, 1,3,3-trimethylpropenylene and 2,3,3-trimethylpropenylene; and

C₂-C₆ alkynylene linking groups containing one or more carbon-carbon triple bonds include ethynylene, propynylene, and 2-butynylene.

A C₁-C₆ haloalkyl or C₁-C₆ haloalkoxy substituent group/moiety will comprise at least one halogen atom, e.g. one, two, three, four or five halogen atoms, examples of which include trifluoromethyl, trifluoromethoxy or pentafluoroethyl.

A C₁-C₆ hydroxyalkyl substituent group/moiety will comprise at least one hydroxyl group, e.g. one, two, three or four hydroxyl groups, examples of which include —CH₂OH, —CH₂CH₂OH, —CH₂CH₂CH₂OH, —CH(OH)CH₂OH, —CH(CH₃)OH and —CH(CH₂OH)₂. The alkyl groups in a di-C₁-C₆ alkylamino group/moiety may be the same as, or different from, one another.

In the definition of R¹⁰, the saturated or unsaturated 3- to 10-membered carbocyclic or heterocyclic ring system may have alicyclic or aromatic properties. An unsaturated ring system will be partially or fully unsaturated.

When R² represents —C(O)-heterocyclyl, the term “heterocyclyl” refers to saturated heterocyclic rings, for example rings containing from 3 to 10 ring atoms, up to four of which are selected from oxygen, nitrogen and sulphur. Particular examples of such heterocyclyl moieties include morpholinyl, azetidinyl, pyrrolidinyl, piperidinyl and piperazinyl.

For the avoidance of doubt, it should be understood that the definitions of the heterocyclic groups/moieties in formula (I) are not intended to include unstable structures or any O—O, O—S or S—S bonds and that a substituent, if present, may be attached to any suitable ring atom.

When any chemical moiety or group in formula (I) is described as being optionally substituted, it will be appreciated that the moiety or group may be either unsubstituted or substituted by one or more of the specified substituents. It will be appreciated that the number and nature of substituents will be selected so as to avoid sterically undesirable combinations.

In formula (I), X¹, X², X³, X⁴ and X⁵ each represent CH (so as to form a phenyl ring) or, alternatively, one or two of X¹, X², X³, X⁴ and X⁵ may additionally represent a nitrogen atom (e.g. to form a pyridyl, pyrazinyl or pyridazinyl ring).

In an embodiment of the invention, X¹, X², X³, X⁴ and X⁵ each represent CH.

In another embodiment, one of X¹, X², X³, X⁴ and X⁵ represents a nitrogen atom and the others represent CH.

In a further embodiment, either X² and X³ each represent a nitrogen atom and X¹, X⁴ and X⁵ each represent CH, or, X³ and X⁴ each represent a nitrogen atom and X¹, X² and X⁵ each represent CH, or, X¹ and X⁴ each represent a nitrogen atom and X², X³ and X⁵ each represent CH, or, X² and X⁵ each represent a nitrogen atom and X¹, X³ and X⁴ each represent CH.

In an embodiment of the invention n is 1 and p is 0 or 1.

Thus, in one aspect, X¹, X², X³, X⁴ and X⁵ each represent CH, n is 1 and p is 0.

In another aspect, X¹, X², X³, X⁴ and X⁵ each represent CH, n is 1 and p is 1.

In still another aspect, X¹, X², X³, X⁴ and X⁵ each independently represent CH or a nitrogen atom, provided that at least one and not more than two of X¹, X², X³, X⁴ and X⁵ simultaneously represent a nitrogen atom, n is 0 and p is 0.

In yet another aspect, X¹, X², X³, X⁴ and X⁵ each independently represent CH or a nitrogen atom, provided that only one of X¹, X², X³, X⁴ and X⁵ represents a nitrogen atom, n is 1 and p is 0.

R¹ represents a halogen atom (e.g. fluorine, chlorine, bromine or iodine) or a methyl or a methoxy group.

In an embodiment of the invention, R¹ represents a fluorine, chlorine or bromine atom, particularly a fluorine atom.

In another embodiment, R¹ represents a methyl group.

In a further embodiment, when n is 1 and X³ represents CH, X³ is substituted by R¹.

In an alternative embodiment, when n is 1 and X⁴ represents CH, X⁴ is substituted by R¹.

R² represents a halogen atom (e.g. fluorine, chlorine, bromine or iodine), —C(O)OCH₃, —C(O)—S—CH₂CN, —C(O)—S—CH₃, —C(O)-heterocyclyl, —SO₂CH₃, a C₂-C₆ or C₂-C₄ alkenyl (e.g. ethenyl) group, or a methyl group optionally substituted by halogen (e.g. fluorine, chlorine, bromine or iodine), hydroxyl, methoxy, —OCH₂CH═CH₂ or —NR⁷R⁸.

In one embodiment, R² represents —C(O)OCH₃, —C(O)—S—CH₂CN, —C(O)—S—CH₃, —C(O)-morpholinyl, —SO₂CH₃, ethenyl, or a methyl group optionally substituted by halogen (e.g. fluorine, chlorine or bromine), hydroxyl, methoxy or —OCH₂CH═CH₂.

In another embodiment, R² represents a methyl group substituted by one hydroxyl group, i.e. —CH₂OH.

In an embodiment of the invention R^{3a} represents a hydrogen atom or a methyl group and R^{3b} represents a hydrogen atom.

In another embodiment of the invention R^{3a} represents a hydrogen atom and R^{3b} represents a fluorine atom.

R⁴ represents —C(O)—S—C(O)N(CH₃)₂, —C(O)CH₂Cl, —C(O)—Y—CH(R¹¹)—R⁹ or —C(O)—CH(R¹¹)—Y—R⁹;

In an embodiment of the invention, R⁴ represents —C(O)—Y—CH(R¹¹)—R⁹ or —C(O)—CH(R¹¹)—Y—R⁹.

In one aspect, R⁵ represents —OCH₂SCH₃, —O—C(O)—R¹⁰, —O—C(O)—NH—R¹⁰, —O—C(O)—O—R¹⁰ or —O—C(O)—S—R¹⁰, in particular an —O—C(O)—R¹⁰ group, and R⁶ represents a hydrogen or a halogen (e.g. fluorine, chlorine, bromine or iodine) atom or a hydroxyl or methyl group, particularly a hydrogen atom or methyl group.

In one embodiment, R⁵ represents a —O—C(O)—R¹⁰ group and R⁶ represents a hydrogen atom or methyl group.

R⁷ and R⁸ each independently represent a hydrogen atom, or a C₁-C₃ alkyl (methyl, ethyl, n-propyl or isopropyl) or a C₁-C₃ hydroxyalkyl (e.g. hydroxymethyl, —(CH₂)₂OH, —(CH₂)₃OH or —CH(CH₂OH)₂) group, or R⁷ and R⁸ together with the nitrogen atom to which they are attached form a 3- to 8-membered, preferably 5- to 6-membered, saturated or partially saturated heterocyclic ring optionally containing a further ring heterogroup selected from nitrogen, S(O)ₘ and oxygen, the heterocyclic ring being optionally substituted by at least one substituent, e.g. one, two, three or four substituents independently, selected from hydroxyl, C₁-C₃ alkyl (methyl, ethyl, n-propyl or isopropyl) and C₁-C₃ hydroxyalkyl (e.g. hydroxymethyl, —(CH₂)₂OH, —(CH₂)₃OH or —CH(CH₂OH)₂).

Examples of 3- to 8-membered saturated or partially saturated heterocyclic rings include morpholine, azetidine, pyrrolidine, piperidine, piperazine, 3-pyrroline, isoindoline, tetrahydroquinoline and thiomorpholine.

In one embodiment, R⁷ and R⁸ each independently represent a hydrogen atom or a C₁-C₂ alkyl (particularly ethyl) or C₂-C₃ hydroxyalkyl group.

In another embodiment, R⁷ and R⁸ together with the nitrogen atom to which they are attached form a 5- to 6-membered saturated or partially saturated heterocyclic ring optionally containing a further ring heterogroup selected from nitrogen, S(O)ₘ and oxygen, the heterocyclic ring being optionally substituted by one or two substituents independently selected from hydroxyl, C₁-C₃ alkyl and C₁-C₃ hydroxyalkyl.

In yet another embodiment, R⁷ and R⁸ together with the nitrogen atom to which they are attached form a 5- to 6-membered saturated heterocyclic ring optionally containing a further ring heterogroup selected from nitrogen, sulphur and oxygen (e.g. pyrrolidinyl, piperidinyl, piperazinyl, thiomorpholinyl or morpholinyl), the heterocyclic ring being optionally substituted by one or two substituents independently selected from hydroxyl, C₁-C₃ alkyl and C₁-C₃ hydroxyalkyl.

In a still further embodiment, R⁷ and R⁸ together with the nitrogen atom to which they are attached form a 5- to 6-membered saturated heterocyclic ring optionally containing a further ring heterogroup selected from nitrogen, sulphur and oxygen, the heterocyclic ring being optionally substituted by one or two substituents independently selected from hydroxyl, methyl and hydroxymethyl.

In an embodiment of the invention, m is 0.

Y represents an oxygen or sulphur atom or a group>NH, particularly an oxygen or sulphur atom.

R⁹ represents hydrogen, halogen (e.g. fluorine, chlorine, bromine or iodine), cyano, —S—CN, —C(O)N(R¹²)₂, C₁-C₆, or C₁-C₄, or C₁-C₂ alkoxycarbonyl, C₁-C₆, or C₁-C₄, or C₁-C₂ alkylcarbonyl (optionally substituted by —OC(O)CH₃), C₁-C₆, or C₁-C₄, or C₁-C₂ alkylcarbonyloxy, C₁-C₆, or C₁-C₄, or C₁-C₂ alkoxy, C₁-C₆, or C₁-C₄, or C₁-C₂ alkylthio, —C(O)—S—C₁-C₆, or C₁-C₄, or C₁-C₂ alkyl, —C(═CH₂)—O—CH₂OCH₃, C₁-C₆, or C₁-C₄, or C₁-C₂ alkyl, C₂-C₆ or C₂-C₄ alkenyl, C₂-C₆ or C₂-C₄ alkynyl or C₃-C₇, or C₅-C₆, cycloalkyl, the latter four groups being optionally substituted by one or more (e.g. one, two, three or four) substituents independently selected from halogen (e.g. fluorine, chlorine, bromine or iodine), hydroxyl, cyano, hydroxymethyl, C₁-C₄, or C₁-C₂, alkoxy and C₁-C₄, or C₁-C₂, alkylcarbonyloxy.

In an embodiment of the invention, R⁹ represents hydrogen, halogen (particularly fluorine), cyano, —S—CN, —C(O)N(R¹²)₂, C₁-C₂ alkoxycarbonyl, C₁-C₂ alkylcarbonyl (optionally substituted by —OC(O)CH₃), C₁-C₂ alkylcarbonyloxy, C₁-C₂ alkoxy, C₁-C₂ alkylthio, —C(O)—S—C₁-C₂ alkyl, —C(═CH₂)—O—CH₂OCH₃, C₁-C₆, or C₁-C₄, or C₁-C₂ alkyl, C₂-C₄ alkenyl, C₂-C₄ alkynyl or C₃-C₆ cycloalkyl, the latter four groups being optionally substituted by one or more (e.g. one, two, three or four) substituents independently selected from halogen (particularly fluorine or chlorine), hydroxyl, cyano, hydroxymethyl, C₁-C₄ alkoxy (particularly methoxy) and C₁-C₄ alkylcarbonyloxy (particularly methylcarbonyloxy).

In another embodiment of the invention, R⁹ represents hydrogen, halogen (particularly fluorine), cyano, methyl, hydroxymethyl or methylcarbonyl.

R¹⁰ represents C₁-C₆, or C₁-C₄, or C₁-C₂ alkyl (optionally substituted by at least one substituent, e.g. one, two, three or four substituents independently, selected from halogen (such as fluorine, chlorine, bromine or iodine), C₁-C₄, or C₁-C₂, alkoxy, C₁-C₄, or C₁-C₂, alkylcarbonyloxy and C₃-C₇, or C₅-C₆, cycloalkyl), or a 3- to 10-membered (e.g. 3-, 4-, 5- or 6- to 7-, 8-, 9- or 10-membered) saturated or unsaturated carbocyclic or heterocyclic ring system optionally substituted by at least one substituent (e.g. one, two, three or four substituents independently) selected from halogen (e.g. fluorine, chlorine, bromine or iodine), carboxyl, hydroxyl, oxo, nitro, cyano, mercapto, C₁-C₆, or C₁-C₄, or C₁-C₂ alkyl, C₂-C₆ or C₂-C₄ alkenyl, C₁-C₆, or C₁-C₄, or C₁-C₂ haloalkyl, C₁-C₆, or C₁-C₄, or C₁-C₂ hydroxyalkyl, C₁-C₆, or C₁-C₄, or C₁-C₂ alkoxy, C₁-C₆, or C₁-C₄, or C₁-C₂ haloalkoxy, C₁-C₆, or C₁-C₄, or C₁-C₂ alkylthio, C₁-C₆, or C₁-C₄, or C₁-C₂ alkylsulphinyl, C₁-C₆, or C₁-C₄, or C₁-C₂ alkylsulphonyl, C₁-C₆, or C₁-C₄, or C₁-C₂ alkylcarbonyl, C₁-C₆, or C₁-C₄, or C₁-C₂ alkylcarbonyloxy, C₁-C₆, or C₁-C₄, or C₁-C₂ alkoxycarbonyl, amino, carboxamido, (mono) C₁-C₆, or C₁-C₄, or C₁-C₂ alkylamino, (di)C₁-C₆, or C₁-C₄, or C₁-C₂ alkylamino and phenyl.

In another embodiment, R¹⁰ represents C₁-C₄, or C₁-C₃, or C₁-C₂ alkyl (optionally substituted by at least one substituent, e.g. one, two, three or four substituents independently, selected from halogen (particularly fluorine), C₁-C₂ alkoxy, C₁-C₂ alkylcarbonyloxy or C₅-C₆ cycloalkyl) or a 3- to 10-membered saturated or unsaturated carbocyclic or heterocyclic ring system optionally substituted as hereinbefore defined.

The heterocyclic ring system will comprise at least one ring heteroatom (e.g. one, two, three or four ring heteroatoms independently) selected from nitrogen, sulphur and oxygen.

Examples of saturated or unsaturated 3- to 10-membered carbocyclic or heterocyclic ring systems that may be used, which may be monocyclic or polycyclic (e.g. bicyclic) in which the two or more rings are fused, include one or more (in any combination) of cyclopropyl, cyclobutyl, cyclopentyl, cyclohexyl, bicyclo[2.2.1]heptyl, cyclopentenyl, cyclohexenyl, phenyl, pyrrolidinyl, piperidinyl, piperazinyl, morpholinyl, thiomorpholinyl, tetrahydrofuranyl, diazabicyclo[2.2.1]hept-2-yl, naphthyl, benzofuranyl, benzothienyl, benzodioxolyl, quinolinyl, oxazolyl, thiadiazolyl (e.g. 1,2,3-thiadiazolyl), 2,3-dihydrobenzofuranyl, tetrahydropyranyl, pyrazolyl, pyrazinyl, thiazolidinyl, indanyl, thienyl, isoxazolyl, pyridazinyl, pyrrolyl, furanyl, thiazolyl, indolyl, imidazolyl, pyrimidinyl, benzimidazolyl, triazolyl, tetrazolyl and pyridinyl. Preferred ring systems include thiadiazolyl, furanyl, thiazolyl, cyclopropyl, cyclobutyl, imidazolyl, oxazolyl, triazolyl, isoxazolyl, thienyl, tetrahydrofuranyl, tetrahydropyranyl and pyrrolyl.

Preferred substituents on the 3- to 10-membered saturated or unsaturated carbocyclic or heterocyclic ring system include alkyl, alkoxy and cyano substituent groups.

In an embodiment of the invention, R¹⁰ represents a 3-, 4- or 5- to 6-, 7- or 8-membered saturated or unsaturated carbocyclic or heterocyclic ring system optionally substituted by one, two, three or four substituents independently selected from halogen, carboxyl, hydroxyl, oxo, nitro, cyano, mercapto, C₁-C₆, or C₁-C₄, or C₁-C₂ alkyl, C₂-C₆ or C₂-C₄ alkenyl, C₁-C₆, or C₁-C₄, or C₁-C₂ haloalkyl, C₁-C₆, or C₁-C₄, or C₁-C₂ hydroxyalkyl, C₁-C₆, or C₁-C₄, or C₁-C₂ alkoxy, C₁-C₆, or C₁-C₄, or C₁-C₂ haloalkoxy, C₁-C₆, or C₁-C₄, or C₁-C₂ alkylthio, C₁-C₆, or C₁-C₄, or C₁-C₂ alkylsulphinyl, C₁-C₆, or C₁-C₄, or C₁-C₂ alkylsulphonyl, C₁-C₆, or C₁-C₄, or C₁-C₂ alkylcarbonyl, C₁-C₆, or C₁-C₄, or C₁-C₂ alkylcarbonyloxy, C₁-C₆, or C₁-C₄, or C₁-C₂ alkoxycarbonyl, amino, carboxamido, (mono)C₁-C₆, or C₁-C₄, or C₁-C₂ alkylamino, (di)C₁-C₆, or C₁-C₄, or C₁-C₂ alkylamino and phenyl.

In another embodiment, R¹⁰ represents a 3- to 6-membered saturated or unsaturated carbocyclic or heterocyclic ring system such as a thiadiazolyl, furanyl, thiazolyl, cyclopropyl, cyclobutyl, imidazolyl, oxazolyl, triazolyl, isoxazolyl, thienyl, tetrahydrofuranyl, tetrahydropyranyl or pyrrolyl ring, the ring system being optionally substituted by at least one substituent (e.g. one, two, three or four, preferably one or two, substituents independently) selected from cyano, C₁-C₄ alkyl (particularly methyl) and C₁-C₄ alkoxy (particularly methoxy).

In still another embodiment, R¹⁰ represents either C₁-C₄, or C₁-C₃, or C₁-C₂ alkyl optionally substituted by C₁-C₂ alkoxy (e.g. methoxymethyl), or a cyclopropyl, oxazolyl or furanyl ring.

In an embodiment of the invention, R¹¹ represents a hydrogen atom.

Examples of compounds of the invention include:
(1R,3aS,3bS,10aR,10bS,11S,12aS)-1-{[(Cyanomethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl methoxyacetate,
(1R,3aS,3bS,10aR,10bS,11S,12aS)-1-{[(Cyanomethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl propanoate,
(1R,3aS,3bS,10aR,10bS,11S,12aS)-1-{[(Fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl propanoate, and
(1R,3aS,3bS,10aR,10bS,11S,12aS)-1-{[(Fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl methoxyacetate,
(1R,3aS,3bS,10aR,10bS,11S,12aS)-1-{[(fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl 1,3-oxazole-4-carboxylate,
(1R,3aS,3bS,10aR,10bS,11S,12aS)-1-{[(cyanomethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl 1,3-oxazole-4-carboxylate,
(1R,3aS,3bS,10aR,10bS,11S,12aS)-1-{[(cyanomethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl methoxyacetate,
(1R,3aS,3bS,10aR,10bS,11S,12aS)-1-{[(fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl cyclopropanecarboxylate,
(1R,3aS,3bS,10aR,10bS,11S,12aS)-1-{[(cyanomethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl cyclopropanecarboxylate,
(1R,3aS,3bS,10aR,10bS,11S,12aS)-1-{[(cyanomethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl propanoate,
(1R,3aS,3bS,10aR,10bS,11S,12aS)-1-{[(fluoromethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl propanoate,
(1R,3aS,3bS,10aR,10bS,11S,12aS)-1-{[(fluoromethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl cyclopropanecarboxylate,
(1R,3aS,3bS,10aR,10bS,11S,12aS)-1-{[(cyanomethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl cyclopropanecarboxylate,
(1R,3aS,3bS,10aR,10bS,11S,12aS)-1-{[(fluoromethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl 1,3-oxazole-4-carboxylate,
(1R,3aS,3bS,10aR,10bS,11S,12aS)-1-{[(cyanomethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl 1,3-oxazole-4-carboxylate,
(1R,3aS,3bS,10aR,11S,12aS)-1-{[(fluoromethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl methoxyacetate,
(1R,2R,3aS,3bS,10aR,10bS,11S,12aS)-1-{[(cyanomethyl)sulfanyl]carbonyl}-11-hydroxy-2,5,10a,12a-tetramethyl-7-phenyl-1,2,3,3a,3b,7,10,10a,10b,11,12,12a-dodecahydrocyclopenta[5,6]naphtho[1,2-f]indazol-1-yl propanoate,
(1R,2R,3aS,3bS,10aR,10bS,11S,12aS)-1-{[(cyanomethyl)sulfanyl]carbonyl}-11-hydroxy-2,5,10a,12a-tetramethyl-7-phenyl-1,2,3,3a,3b,7,10,10a,10b,11,12,12a-dodecahydrocyclopenta[5,6]naphtho[1,2-f]indazol-1-yl furan-2-carboxylate,
   and pharmaceutically acceptable salts of any one thereof.

It should be noted that each of the chemical compounds listed above represents a particular and independent aspect of the invention.

The present invention further provides a process for the preparation of a compound of formula (I) or a pharmaceutically acceptable salt thereof as defined above which comprises (i) reacting a compound of formula (II)

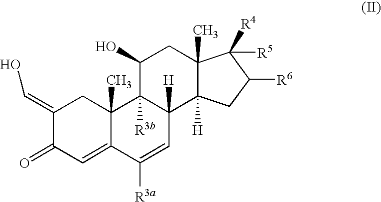

wherein R^{3a}, R^{3b}, R⁴, R⁵ and R⁶ are as defined in formula (I), with a compound of formula (III) or an acid addition salt (e.g. hydrochloride salt) thereof

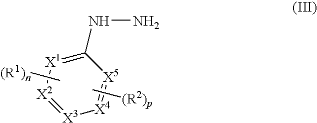

wherein n, p, R¹, R², X¹, X², X³, X⁴ and X⁵ are as defined in formula (I); or
(ii) when R⁴ represents —C(O)—Y—CH(R¹¹)—R⁹ and Y represents a sulphur atom, reacting a compound of formula (IV)

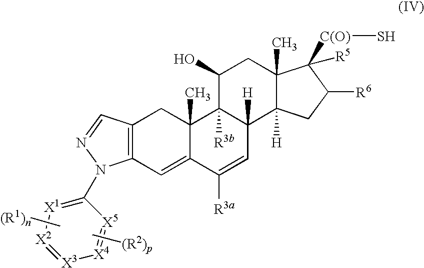

where n, p, X¹, X², X³, X⁴, X⁵, R¹, R², R^{3a}, R^{3b}, R⁵ and R⁶ are as defined in formula (I), with a compound of formula (V), R⁹—CH(R¹¹)-L, where L represents a leaving group (e.g. a halogen atom) and R⁹ and R¹¹ are as defined in formula (I);
and optionally thereafter carrying out one or more of the following procedures:
converting a compound of formula (I) into another compound of formula (I)
removing any protecting groups
forming a pharmaceutically acceptable salt.

Process (i) above is conveniently carried out in the presence of an organic solvent such as acetic acid/water mixture at room temperature (20° C.) or, alternatively, in the presence of an organic solvent such as ethanol at a temperature in the range from room temperature (20° C.) to 90° C. Preferably, the reaction is carried out in the presence of a base, e.g. an alkali metal acetate such as potassium acetate.

The process (ii) above is conveniently carried out in the presence of an organic solvent such as dichloromethane, N,N-dimethylformamide or acetone in the presence of a base (e.g. Hünig's base or an alkali metal base such potassium carbonate, sodium carbonate or sodium hydrogen carbonate) at a temperature in the range from, for example, 25° C. to 35° C.

The compounds of formula (II) may be prepared by reacting a compound of formula (X)

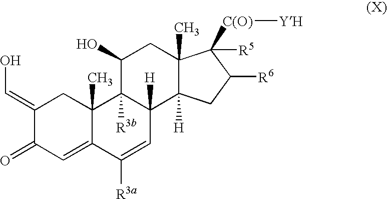

wherein Y′ represents an oxygen or sulphur atom and R^{3a}, R^{3b}, R⁵ and R⁶ are as defined in formula (II), with L¹-C(O)N(CH₃)₂, or chloromethane, or a compound of formula (V) above optionally followed by reaction with an amine of formula (XI), R⁹—CH(R¹¹)—NH₂, to obtain compounds of formula (II) in which R⁴ is —C(O)—Y—CH(R¹¹)—R⁹ where Y is NH, or with R⁹—Y—CH(R¹¹)-L² (formula XIA), wherein L¹ and L² are leaving groups (e.g. halogen atoms) and R⁹ and R¹¹ are as defined in formula (I).

Compounds of formula (X) may be prepared by reacting a compound of formula (XII)

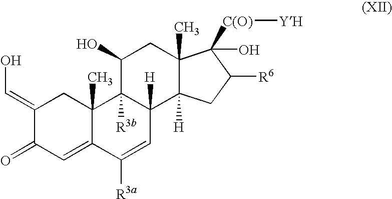

wherein R^{3a}, R^{3b}, R⁶ and Y′ are as defined in formula (X), with L³-CH₂SCH₃ (formula XV), L³-C(O)—R¹⁰ (formula XVI), L³-C(O)—NH—R¹⁰ (formula XVII), L³-C(O)—O—R¹⁰ (formula XVIII) or L³-C(O)—S—R¹⁰ (formula XIX) where L³ represents a leaving group and R¹⁰ is as defined in formula (I).

Compounds of formula (XII) in which Y′ is sulphur may be prepared by reacting a corresponding compound of formula (XII) in which Y′ is oxygen with hydrogen sulphide according to methods known in the art.

Compounds of formula (XII) in which Y′ is oxygen may be prepared by reacting a compound of formula (XIII)

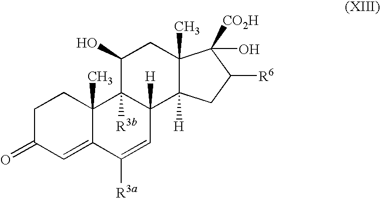

wherein R^{3a}, R^{3b} and R⁶ are as defined in formula (XII), with methyl or ethyl formate in the presence of a base such as sodium hydride, in a manner analogous to the method described in the journal article by Wuest, F. et al., Steroids, 68 (2003), 177-191.

Compounds of formula (XIII) may be prepared from compounds of formula (XIV)

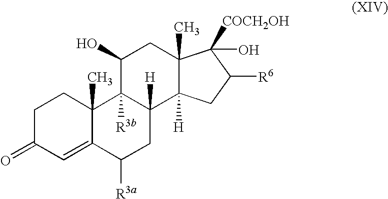

wherein R^{3a}, R^{3b}, R⁶ are as defined in formula (XIII), by introducing a suitable protecting group on the —C(O)CH₂OH group, followed by a dehydrogenation reaction to form a carbon-carbon double bond in the 6,7 position, then followed by removal of the protecting group and lastly by an oxidative degradation reaction, all such reaction steps being carried out according to processes known in the art.

Compounds of formula (IV) may be prepared by reacting a compound of formula (X) as defined above in which Y′ is oxygen with a compound of formula (III) as defined above, followed by reaction with hydrogen sulphide to convert Y′ from oxygen to sulphur according to methods known in the art.

Alternatively, compounds of formula (IV) may be prepared by reacting a compound of formula (XII) in which Y′ is oxygen with a compound of formula (III) as defined above, followed by reaction with hydrogen sulphide to convert Y′ from oxygen to sulphur, and then followed by reaction with a compound of formula (XV) to (XIX).

Compounds of formulae (III), (V), (XI), (XIA), (XIV), (XV), (XVI), (XVII), (XVIII) and (XIX) are either commercially available, are well known in the literature or may be prepared easily using known techniques.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, trifluoroacetate, sulfate, phosphate, acetate, fumarate, maleate, tartrate, lactate, citrate, pyruvate, succinate, oxalate, methanesulphonate or p-toluenesulphonate.

The compounds of formula (I) and pharmaceutically acceptable salts thereof may exist in solvated, for example hydrated, as well as unsolvated forms, and the present invention encompasses all such solvated forms.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically and diastereomerically pure forms are particularly desired.

The compounds of formula (I) and their pharmaceutically acceptable salts have activity as pharmaceuticals, in particular as modulators of glucocorticoid receptor activity, and thus may be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis;cutaneous lymphomas, non-melanoma skin cancer and other dysplastic lesions; drug-induced disorders including fixed drug eruptions;
3. eyes: blepharitis; conjunctivitis, including perennial and vernal allergic conjunctivitis; iritis; anterior and posterior uveitis; choroiditis; autoimmune, degenerative or inflammatory disorders affecting the retina; ophthalmitis including sympathetic ophthalmitis; sarcoidosis; infections including viral, fungal, and bacterial;
4. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
5. allograft rejection: acute and chronic following, for example, transplantation of kidney, heart, liver, lung, bone marrow, skin or cornea or following blood transfusion; or chronic graft versus host disease;
6. other auto-immune and allergic disorders including rheumatoid arthritis, irritable bowel syndrome, systemic lupus erythematosus, multiple sclerosis, Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome and Sazary syndrome;
7. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
8. infectious diseases: virus diseases such as genital warts, common warts, plantar warts, hepatitis B, hepatitis C, herpes simplex virus, molluscum contagiosum, variola, human immunodeficiency virus (HIV), human papilloma virus (HPV), cytomegalovirus (CMV), varicella zoster virus (VZV), rhinovirus, adenovirus, coronavirus, influenza, para-influenza; bacterial diseases such as tuberculosis and mycobacterium avium, leprosy; other infectious diseases, such as fungal diseases, chlamydia, candida, aspergillus, cryptococcal meningitis, pneumocystis carni, cryptosporidiosis, histoplasmosis, toxoplasmosis, trypanosome infection and leishmaniasis.

Thus, the present invention provides a compound of formula (I) or a pharmaceutically acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In particular, the compounds of the invention (including pharmaceutically acceptable salts) may be used in the treatment of asthma {such as bronchial, allergic, intrinsic, extrinsic or dust asthma, particularly chronic or inveterate asthma (for example late asthma or airways hyper-responsiveness)}, chronic obstructive pulmonary disease (COPD) or allergic rhinitis.

The invention also provides a method of treating, or reducing the risk of, an obstructive airways disease or condition (e.g. asthma or COPD) which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

Dry powder formulations and pressurized HFA aerosols of the compounds of the invention (that is, compounds of formula (I) and pharmaceutically acceptable salts thereof) may be administered by oral or nasal inhalation. For inhalation, the compound is desirably finely divided. The finely divided compound preferably has a mass median diameter of less than 10 micrometres (μm), and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention (that is, compounds of formula (I) and pharmaceutically acceptable salts thereof) may also be administered in conjunction with other compounds used for the treatment of the above conditions.

The invention therefore further relates to combination therapies wherein a compound of the invention or a pharmaceutical composition or formulation comprising a compound of the invention is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention and a receptor antagonist for leukotrienes (LT) B4, LTC4, LTD4, and LTE4 selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention and a proton pump inhibitor (such as omeprazole) or a gastroprotective histamine type 2 receptor antagonist.

The present invention further relates to the combination of a compound of the invention and an antagonist of the histamine type 4 receptor.

The present invention still further relates to the combination of a compound of the invention and an alpha-1/alpha-2 adrenoreceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention and a beta-adrenoreceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention and a chromone, such as sodium cromoglycate or nedocromil sodium.

The present invention further relates to the combination of a compound of the invention with an agent that modulates a nuclear hormone receptor such as PPARs.

The present invention still further relates to the combination of a compound of the invention together with an immunoglobulin (Ig) or Ig preparation or an antagonist or antibody modulating Ig function such as anti-IgE (for example omalizumab).

The present invention further relates to the combination of a compound of the invention and another systemic or topically-applied anti-inflammatory agent, such as thalidomide or a derivative thereof, a retinoid, dithranol or calcipotriol.

The present invention still further relates to the combination of a compound of the invention and combinations of aminosalicylates and sulfapyridine such as sulfasalazine, mesalazine, balsalazide, and olsalazine; and immunomodulatory agents such as the thiopurines.

The present invention further relates to the combination of a compound of the invention together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention and a cardiovascular agent such as a calcium channel blocker, a beta-adrenoreceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention together with a parenterally or topically-applied (including inhaled) local anaesthetic agent such as lignocaine or a derivative thereof.

A compound of the present invention can also be used in combination with an anti-osteoporosis agent including a hormonal agent such as raloxifene, or a biphosphonate such as alendronate.

The present invention still further relates to the combination of a compound of the invention together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2×7; (xxvii) inhibitor of transcription factor activation such as NFkB, API, or STATS; or (xxviii) a glucocorticoid receptor agonist.

In a further aspect the present invention provides a (fixed dose) combination (for example for the treatment of COPD, asthma or allergic rhinitis) of a compound of formula (I) or a pharmaceutically acceptable salt thereof as hereinbefore defined, one or more agents independently selected from:
a selective β₂ adrenoreceptor agonist (such as metaproterenol, isoproterenol, isoprenaline, albuterol, salbutamol, formoterol, salmeterol, terbutaline, orciprenaline, bitolterol mesylate, pirbuterol or indacaterol);
a phosphodiesterase inhibitor (such as a PDE4 inhibitor);
a protease inhibitor (such as a neutrophil elastase or matrix metalloprotease MMP-12 inhibitor);
an anticholinergic agent;
a modulator of chemokine receptor function (such as a CCR1 receptor antagonist); and
an inhibitor of kinase function (such as the kinases p38 or IKK);
   and optionally one or more pharmaceutically acceptable excipients.

The invention also provides a pharmaceutical product comprising a preparation of a first active ingredient which is a compound of formula (I) or a pharmaceutically acceptable salt thereof as hereinbefore defined, and a preparation of a second active ingredient which is:
a selective β₂ adrenoreceptor agonist;
a phosphodiesterase inhibitor;
a protease inhibitor;
an anticholinergic agent;
a modulator of chemokine receptor function; or
an inhibitor of kinase function;
   wherein the preparations are for simultaneous, sequential or separate administration to a patient in need thereof.

In another aspect, the invention provides a kit comprising a preparation of a first active ingredient which is a compound of formula (I) or a pharmaceutically acceptable salt thereof as hereinbefore defined, and a preparation of a second active ingredient which is:
a selective β₂ adrenoreceptor agonist;
a phosphodiesterase inhibitor;
a protease inhibitor;
an anticholinergic agent;
a modulator of chemokine receptor function; or
an inhibitor of kinase function;
   and instructions for the simultaneous, sequential or separate administration of the preparations to a patient in need thereof.

A compound of the invention can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will now be further explained by reference to the following illustrative examples in which the following abbreviations are used:
EtOAc ethyl acetate
HCl hydrochloric acid
H₂S hydrogen sulphide
CH₂Cl₂ dichloromethane (DCM)
DMF N,N-dimethylformamide
NaH sodium hydride
MgSO₄ magnesium sulfate
NaNO₂ sodium nitrite
K₂CO₃ potassium carbonate
SnCl₂ tin (II) chloride
NaOH sodium hydroxide
Na₂SO₄ sodium sulfate
NH₄Cl ammonium chloride
DIEA diisopropylethylamine
DME dimethyl ether
DMSO dimethylsulfoxide
EtOH ethanol
THF tetrahydrofuran
TFA trifluoroacetic acid
HCl hydrochloric acid
DCM dichloromethane
NaHCO₃ sodium hydrogen carbonate
Et₃N triethylamine
MeOH methanol
MeCN/CH₃CN acetonitrile
EDTA ethylenediaminetetraacetic acid
conc. concentrated
rt room temperature
h hours
min minutes
M molar
MS mass spectrometry
APCI atmospheric chemical ionisation method
ESI electron spray ionisation method
NMR nuclear magnetic resonance
SCX solid phase extraction with a sulfonic acid sorbent
HPLC high performance liquid chromatography
LC-MS liquid chromatography with mass spectrometry detection

### General Methods

NMR spectra were recorded on a Varian Mercury-VX 300 MHz instrument or a Varian Inova 400 MHz instrument. The central peaks of chloroform-d (H 7.26 ppm), acetone-d₆ (H 2.05 ppm), acetonitrile-d₃ (δ_{H} 1.94 ppm) or DMSO-d₆ (H 2.50 ppm) were used as internal references.

The following method was used for LC/MS analysis:

Instrument Agilent 1100; Column Waters Symmetry 2.1×30 mm; Mass APCI; Flow rate 0.7 mL/min; Wavelength 254 nm; Solvent A: water+0.1% TFA; Solvent B: acetonitrile+0.1% TFA; Gradient 15-95%/B 2.7 min, 95% B 0.3 min.

Column chromatography was carried out using silica gel (0.040-0.063 mm, Merck). For preparative HPLC either a Kromasil® KR-100-5-C18 column (250×20 mm, Akzo Nobel) and mixtures of acetonitrile/water (0.1% TFA) at a flow rate of 10 ml/min or a XTerra® Prep MS C₁₈ OBD™ Column, 5 μm, 19×50 mm (acetonitrile/water/0.1% NH₃) at a flow rate of 20 ml/min was used. UV=254 nm or 220 nm was used for detection.

Unless stated otherwise, starting materials were commercially available. All solvents and commercial reagents were of laboratory grade and were used as received.

### Intermediate 1

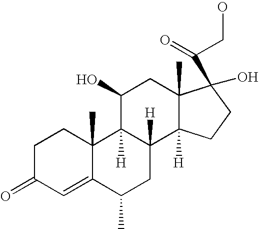

### (6S,8S,9S,10R,11S,13S,14S,17R)-11,17-Dihydroxy-17-(2-hydroxyacetyl)-6,10,13-trimethyl-6,7,8,9,10,11,12,13,14,15,16,17-dodecahydro-1H-cyclopenta[a]phenanthren-3(2H)-one

In a 250 mL round-bottomed flask was suspended (6S,8S,9S,10R,11S,13S,14S,17R)-11,17-dihydroxy-17-(2-hydroxyacetyl)-6,10,13-trimethyl-6,7,8,9,10,11,12,13,14,15,16,17-dodecahydro-3H-cyclopenta[a]phenanthren-3-one (6α-Methylprednisolon, 4.3 g, 11.48 mmol) in EtOAc (80 mL) and the reaction mixture was diluted with ethanol (20.0 mL). Tris(triphenylphosphine)rhodium(I) chloride (Wilkinson's catalyst, 1 g, 1.08 mmol) was added together with a magnetic stirrer bar. The mixture was vigorously stirred in a hydrogen atmosphere (1 atm) at room temperature for 1 week. The mixture was filtered through a glass sinter and the filtrate was concentrated in vacuo, giving 4.07 g of a light brown solid. The material was used as such without any further purification. APCI-MS m/z: 377 [MH⁺].

### Intermediate 2

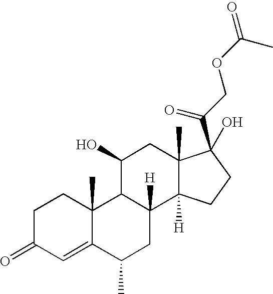

### 2-((6S,8S,9S,10R,11S,13S,14S,17R)-11,17-Dihydroxy-6,10,13-trimethyl-3-oxo-2,3,6,7,8,9,10,11,12,13,14,15,16,17-tetradecahydro-1H-cyclopenta[a]phenanthren-17-yl)-2-oxoethyl acetate

In a 100 mL round-bottomed flask intermediate 1 (1.05 g, 2.79 mmol) was suspended in ethyl acetate (20 mL) and pyridine (2 mL, 24.78 mmol) followed by acetic anhydride (2 mL, 21.16 mmol) were added. The mixture was stirred for four hours at 55° C. and overnight at room temperature. The crude mixture was diluted with ethyl acetate (30 ml) and washed with 1M HCl (30 ml) and brine (20 ml). Drying over Na₂SO₄ was followed by filtration and evaporation of the solvent in vacuo yielded a yellowish solid which was washed with a minor amount of DCM (5-10 ml) and the resulting solid was dried in a stream of air on the sinter to give 0.85 g of the desired compound as a white solid. APCI-MS m/z: 419 [MH⁺].

### Intermediate 3

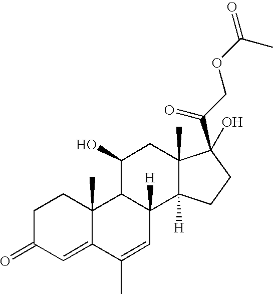

### 2-((8S,9S,10R,11S,13S,14S,17R)-11,17-Dihydroxy-6,10,13-trimethyl-3-oxo-2,3,8,9,10,11,12,13,14,15,16,17-dodecahydro-1H-cyclopenta[a]phenanthren-17-yl)-2-oxoethyl acetate

In a 20 mL Vial was added intermediate 2 (0.25 g, 0.60 mmol) and 2,3,5,6-tetrachlorocyclohexa-2,5-diene-1,4-dione (Chloranil) (0.5 g, 2.03 mmol) in EtOAc (6.2 mL) and acetic acid (1.25 mL) to give a suspension. To the mixture was added a magnetic stirrer bar and the mixture was degassed with N₂. The sealed vial was heated whilst stirring at 85° C. overnight and was then allowed to cool to room temperature. The mixture was filtered, and the resulting solid was washed with EtOAc (20 ml). The filtrate was diluted with EtOAc up to a volume of 50 ml in a separation funnel and the organic solution was washed with a Na₂CO₃ solution (40 ml). The aqueous phase was extracted with another portion of EtOAc (20 ml) and the combined organic phases were washed with 1M NaOH (2×20 ml), water (20 ml) and brine (20 ml). Drying over Na₂SO₄, filtration and evaporation of the solvent in vacuo resulted in a greenish residue which was redissolved in DCM (20 ml) and the solution was evaporated to dryness to give 0.27 g of the desired compound as a brownish, dry foam. APCI-MS m/z: 417 [MH⁺].

### Intermediate 4

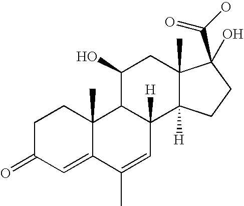

### (8S,9S,10R,11S,13S,14S,17R)-11,17-Dihydroxy-6,10,13-trimethyl-3-oxo-2,3,8,9,10,11,12,13,14,15,16,17-dodecahydro-1H-cyclopenta[a]phenanthrene-17-carboxylic acid

In a 100 mL round-bottomed flask was dissolved intermediate 3 (0.25 g, 0.60 mmol) in MeOH (30 mL) and 1M sodium hydroxide (6 mL, 6.00 mmol) was added. The open flask was vigorously stirred in an air atmosphere for 3 hours followed by acidification with 1M HCl (7 ml). The organic solvent was removed in vacuo and the obtained wet residue was extracted with EtOAc (2×20 ml). The combined organic extracts were washed with brine (15 ml) and dried over Na₂SO₄. Filtration and evaporation of the solvent in vacuo resulted in 220 mg of the desired compound as an orange foam. APCI-MS m/z: 361 [MH⁺].

### Intermediate 5

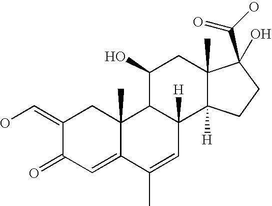

### (8S,9S,10R,11S,13S,14S,17R,Z)-11,17-Dihydroxy-2-(hydroxymethylene)-6,10,13-trimethyl-3-oxo-2,3,8,9,10,11,12,13,14,15,16,17-dodecahydro-1H-cyclopenta[a]phenanthrene-17-carboxylic acid

To a stirred suspension of sodium hydride (60% in mineral oil, 0.244 g, 6.10 mmol) in THF (6 mL) under argon was added intermediate 4 (0.22 g, 0.61 mmol) in small portions followed by ethyl formate (2.484 mL, 30.52 mmol). Stirring was continued at room temperature. The suspension was stirred at the same temperature for 2 hours. The reaction was quenched by careful addition of 2M NaOH (5 ml), the phases were separated in a separation funnel and the aqueous phase was collected. The organic phase was extracted with additional 2M NaOH (2×5 ml) and the combined aqueous solutions were diluted with water (15 ml) and washed with EtOAc (20 ml). The aqueous phase was acidified with 4M HCl, and the product was extracted with EtOAc (2×20 ml). The combined organic phases were washed with brine (10 ml) and dried over Na₂SO₄. Filtration and evaporation of the solvent in vacuo gave a slightly yellowish semi-solid (0.19 g) which was used in the next step without any further purification. APCI-MS m/z: 389 [MH⁺].

### Intermediate 6

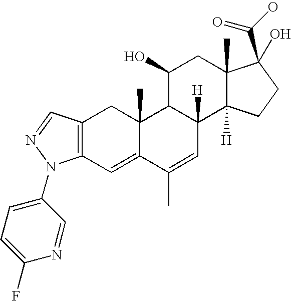

### (1R,3aS,3bS,10aR,11S,12aS)-7-(6-Fluoropyridin-3-yl)-1,11-dihydroxy-5,10a,12a-trimethyl-1,2,3,3a,3b,7,10,10a,10b,11,12,12a-dodecahydrocyclopenta[5,6]naphtho[1,2-f]indazole-1-carboxylic acid

In a 25 mL round-bottomed flask was dissolved intermediate 5 (0.19 g, 0.49 mmol) in acetic acid (5 mL) to give an orange solution. The solution was degassed with nitrogen gas. To this solution was added 2-fluoro-5-hydrazinylpyridine (0.062 g, 0.49 mmol) at room temperature. The mixture was stirred for an additional 30 minutes at the same temperature after which the mixture was freeze-dried to yield 0.23 g of the desired compound as an orange solid. APCI-MS m/z: 480 [MH⁺].

### Intermediate 7

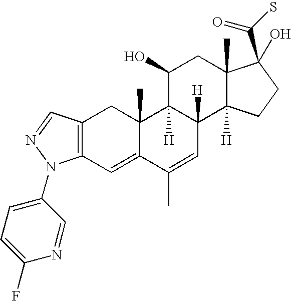

### (1R,3aS,3bS,10aR,10bS,11S,12aS)-7-(6-Fluoropyridin-3-yl)-1,11-dihydroxy-5,10a,12a-trimethyl-1,2,3,3a,3b,7,10,10a,10b,11,12,12a-dodecahydrocyclopenta[5,6]naphtho[1,2-f]indazole-1-carbothioic S-acid

In a 50 mL round-bottomed flask was dissolved intermediate 6 (0.23 g, 0.48 mmol) and di(1H-imidazol-1-yl)methanone (0.233 g, 1.44 mmol) in DMF (5 mL) and the mixture was stirred in a sealed flask overnight at room temperature. Hydrogen sulfide was bubbled through the solution for 3 minutes and the resulting solution was allowed to stir for an additional 5 minutes. The resulting solution was added to 50 ml 1M HCl and the obtained mixture was extracted with EtOAc (2×20 ml). The combined organic phases were washed with 1M HCl (2×15 ml) and brine (10 ml) and were subsequently dried over Na₂SO₄. Filtration and evaporation of the solvent in vacuo yielded 0.19 g of the desired compound as an orange solid. APCI-MS m/z: 496 [MH⁺].

### Intermediate 8

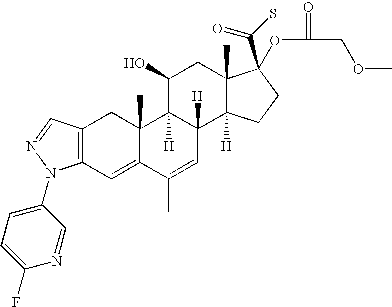

### (1R,3aS,3bS,10aR,10bS,11S,12aS)-7-(6-Fluoropyridin-3-yl)-11-hydroxy-1-[(methoxyacetyl)oxy]-5,10a,12a-trimethyl-1,2,3,3a,3b,7,10,10a,10b,11,12,12a-dodecahydrocyclopenta[5,6]naphtho[1,2-f]indazole-1-carbothioic S-acid

In a 50 mL round-bottomed flask was dissolved intermediate 7 (0.092 g, 0.19 mmol) and triethylamine (0.064 mL, 0.46 mmol) in DCM (10 mL) and 2-methoxyacetyl chloride (0.042 g, 0.39 mmol) was added at room temperature. The mixture was stirred for an additional 10 minutes and subsequently, N₁-ethyl-N₂,N₂-dimethylethane-1,2-diamine (0.087 mL, 0.56 mmol) was added. The mixture was stirred for another 10 minutes and subsequently diluted with DCM to a total volume of 25 ml. Having washed with 1M HCl (2×20 ml) and brine (10 ml), the organic phase was dried over Na₂SO₄, filtered and the solvent was evaporated in vacuo to yield 110 mg of the target compound as a yellow solid. APCI-MS m/z: 568 [MH⁺].

### Intermediate 9

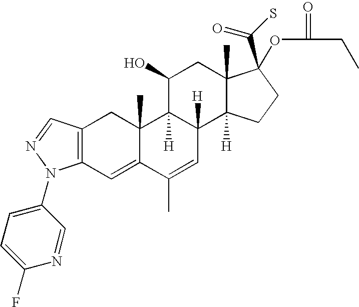

### (1R,3aS,3bS,10aR,10bS,11S,12aS)-7-(6-Fluoropyridin-3-yl)-11-hydroxy-5,10a,12a-trimethyl-1-(propanoyloxy)-1,2,3,3a,3b,7,10,10a,10b,11,12,12a-dodecahydrocyclopenta[5,6]naphtho[1,2-f]indazole-1-carbothioic S-acid

The compound was prepared according to the description for Intermediate 8, starting from Intermediate 7. APCI-MS m/z: 552 [MH⁺].

### Intermediate 10

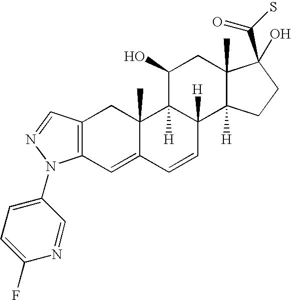

### (1R,3aS,3bS,10aR,10bS,11S,12aS)-7-(6-Fluoropyridin-3-yl)-1,11-dihydroxy-10a,12a-dimethyl-1,2,3,3a,3b,7,10,10a,10b,11,12,12a-dodecahydrocyclopenta[5,6]naphtho[1,2-f]indazole-1-carbothioic S-acid

The compound was prepared according to the sequence and methods described for intermediates 2-7, starting from Hydrocortisone. APCI-MS m/z: 482 [MH⁺].

### Intermediate 11

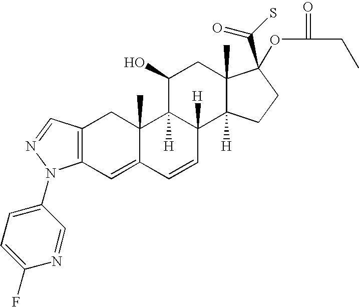

### (1R,3aS,3bS,10aR,10bS,11S,12aS)-7-(6-Fluoropyridin-3-yl)-11-hydroxy-10a,12a-dimethyl-1-(propanoyloxy)-1,2,3,3a,3b,7,10,10a,10b,11,12,12a-dodecahydrocyclopenta[5,6]naphtho[1,2-f]indazole-1-carbothioic S-acid

The compound was prepared according to the description for Intermediate 8, starting from Intermediate 10. APCI-MS m/z: 538 [MH⁺].

### Intermediate 12

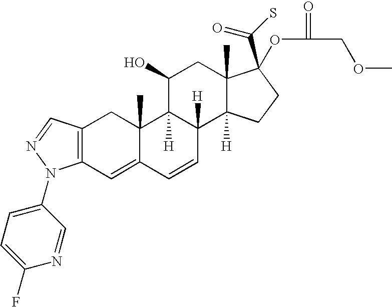

### (1R,3aS,3bS,10aR,10bS,11S,12aS)-7-(6-Fluoropyridin-3-yl)-11-hydroxy-1-[(methoxyacetyl)oxy]-10a,12a-dimethyl-1,2,3,3a,3b,7,10,10a,10b,11,12,12a-dodecahydrocyclopenta[5,6]naphtho[1,2-f]indazole-1-carbothioic S-acid

The compound was prepared according to the description for Intermediate 8, starting from Intermediate 10. APCI-MS m/z: 554 [MH⁺].

### EXAMPLE 1

### (1R,3aS,3bS,10aR,10bS,11S,12aS)-1-{[(Cyanomethyl)sulfanyl]carbonyl}-7-(6-fluoropyridin-3-yl)-11-hydroxy-5,10a,12a-trimethyl-1,2,3,3a,3b,7,10,10a,10b,-11,12,12a-dodecahydrocyclopenta[5,6]naphtho[1,2-f]indazol-1-yl methoxyacetate

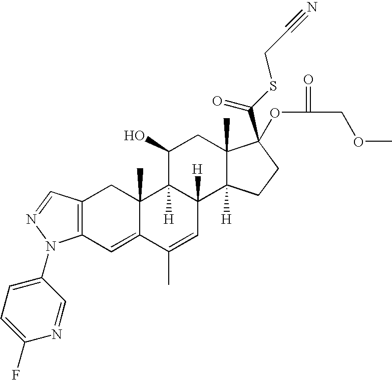

In a vial was dissolved intermediate 8 (0.05 g, 0.09 mmol) and N,N-diisopropyl-ethylamine (0.020 mL, 0.12 mmol) in dioxane (3 mL). 2-Bromoacetonitrile (7.04 μL, 0.11 mmol) was added at room temperature and the mixture was stirred for 10 minutes. The reaction mixture was concentrated in vaccuo and the obtained residue was redissolved in CH₃CN (3 ml) and water (1 ml) was added. The obtained solution was filtered and injected onto a preparative HPLC-column. The product containing fractions were combined and freeze-dried to give 9 mg of the desired compound as a white solid. APCI-MS m/z: 607 [MH⁺].

¹H NMR (400 MHz, CDCl₃) δ 8.39 (1H, s), 8.03 (1H, m), 7.54 (1H, s), 7.10 (1H, dd), 6.25 (1H, s), 5.76 (1H, s), 4.55 (1H, bs), 4.06 (2H, s), 3.81 (1H, d, AB), 3.59 (1H, d, AB), 3.45 (3H, s), 3.20-2.99 (2H, m), 2.82-2.64 (2H, m), 2.17-1.92 (4H, m), 1.87 (3H, s), 1.86-1.76 (1H, m), 1.69-1.56 (1H, m), 1.49 (1H, d), 1.34 (1H, m), 1.21 (4H, s), 1.07 (3H, s)

### EXAMPLE 2

### (1R,3aS,3bS,10aR,10bS,11S,12aS)-1-{[(Cyanomethyl)sulfanyl]carbonyl}-7-(6-fluoropyridin-3-yl)-11-hydroxy-5,10a,12a-trimethyl-1,2,3,3a,3b,7,10,10a,10b,-11,12,12a-dodecahydrocyclopenta[5,6]naphtho[1,2-f]indazol-1-yl propanoate

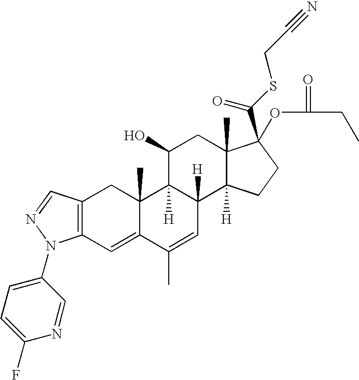

The compound was prepared according to the description in Example 1, starting from Intermediate 9. APCI-MS m/z: 591 [MH⁺].

¹H NMR (400 MHz, CDCl₃) δ 8.39 (1H, s), 8.03 (1H, m), 7.55 (1H, s), 7.10 (1H, dd), 6.25 (1H, s), 5.77 (1H, s), 4.56 (1H, bs), 3.80 (1H, d, AB), 3.57 (1H, d, AB), 3.09-2.96 (2H, m), 2.80-2.66 (2H, m), 2.37 (2H, dq), 2.14 (1H, d), 2.08-1.94 (3H, m), 1.88 (3H, s), 1.88-1.78 (1H, m), 1.66-1.56 (1H, m), 1.50 (1H, d), 1.22 (4H, s), 1.16 (3H, t), 1.05 (3H, s)

### EXAMPLE 3

### (1R,3aS,3bS,10aR,10bS,11S,12aS)-1-{[(Fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-ylpropanoate

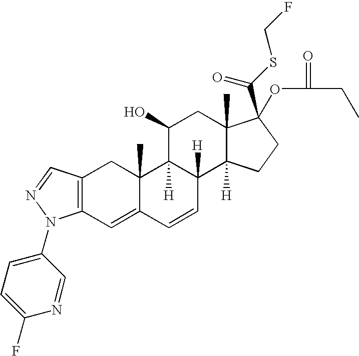

In a 50 mL round-bottomed flask was dissolved intermediate 11 (0.055 g, 0.10 mmol) and N,N-diisopropylethylamine (0.051 mL, 0.31 mmol) in dioxane (3 mL). A 60% solution of bromofluoromethane (0.039 g, 0.20 mmol) in DMF was added at room temperature. The mixture was stirred for 60 minutes and the mixture was concentrated in vacuo. The obtained residue was redissolved in CH₃CN (3 ml) and water (1 ml) was added. The obtained solution was filtered and injected onto a preparative HPLC-column. The product containing fractions were combined and freeze-dried to yield 6 mg of the desired compound as a white solid. APCI-MS m/z: 570 [MH⁺].

¹H NMR (400 MHz, CDCl₃) δ 8.39 (1H, s), 8.01 (1H, m), 7.54 (1H, s), 7.08 (1H, dd), 6.13 (1H, d), 6.12 (1H, s), 6.07-5.62 (3H, m), 4.56 (1H, bs), 3.11-2.99 (2H, m), 2.79 (1H, t), 2.70 (1H, d), 2.38 (2H, dq), 2.17 (1H, d), 2.08-1.93 (3H, m), 1.92-1.80 (1H, m), 1.65-1.57 (1H, m), 1.52 (1H, d), 1.24 (3H, s), 1.19-1.14 (3H+1H, t+bs), 1.05 (3H, s)

### EXAMPLE 4

### (1R,3aS,3bS,10aR,10bS,11S,12aS)-1-{[(Fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl methoxyacetate

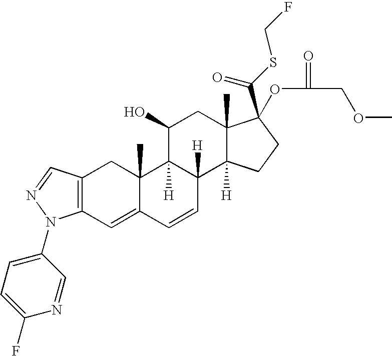

The compound was prepared according to the description in Example 3, starting from Intermediate 12. APCI-MS m/z: 586 [MH⁺].

¹H NMR (400 MHz, CDCl₃) δ 8.38 (1H, s), 8.01 (1H, m), 7.54 (1H, s), 7.09 (1H, dd), 6.13 (1H, d), 6.12 (1H, s), 6.06-5.64 (3H, m), 4.55 (1H, bs), 4.07 (2H, s), 3.46 (3H, s), 3.09 (1H, m), 3.02 (1H, d), 2.80 (1H, t), 2.69 (1H, d), 2.18-1.94 (4H, m), 1.90-1.78 (1H, m), 1.69-1.58 (1H, m), 1.52 (1H, d), 1.25 (3H, s), 1.18 (1H, bs), 1.06 (3H, s)

### EXAMPLES 5 to 18

The compounds of Examples 5 to 18 were prepared by processes analogous to those described in Examples 1 to 4 or by processes analogous to those known in the art. The compounds of Examples 17 and 18 were prepared starting from cortivazol.

| | Ex. | | | APCI |
|---|---|---|---|---|
| Structure | No. | Compound | ¹H NMR | m/z |
| 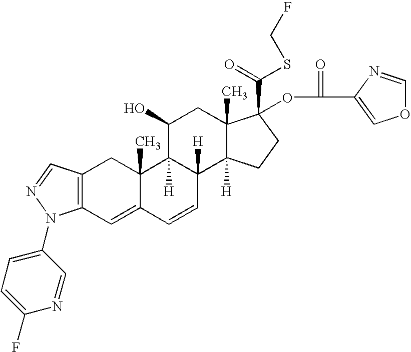 | 5 | (1R,3aS,3bS,10aR,10bS,11S,12aS)-1- {[(fluoromethyl)sulfanyl]carbonyl}-7- (6-fluoropyridin-3-yl)-11-hydroxy- 10a,12a-dimethyl- 1,2,3,3a,3b,7,10,10a,10b,11,12,12a- dodecahydrocyclopenta[5,6]naphtho [1,2-f]indazol-1-yl 1,3-oxazole-4- carboxylate | 1H NMR (399.99 MHz, CdCl₃) δ 8.39 (1H, bs), 8.31 (1H, d, J = 0.91 Hz), 8.02 (1H, m), 7.95 (1H, d, J = 0.91 Hz), 7.55 (1H, s), 7.08 (1H, dd), 6.15-6.09 (2H, m), 6.07-5.62 (3H, m), 4.62 (1H, bs), 3.16 (1H, m), 3.04 (1H, d), 2.83 (1H, t), 2.74 (1H, d), 2.31 (1H, dd), 2.25-2.15 (1H, m), 2.11 (1H, dd), 2.07- 1.92 (2H, m), 1.71-1.57 (2H, m), 1.26 (3H, s), 1.22 (1H, bs), 1.11 (3H, s) | 609.2 |
| 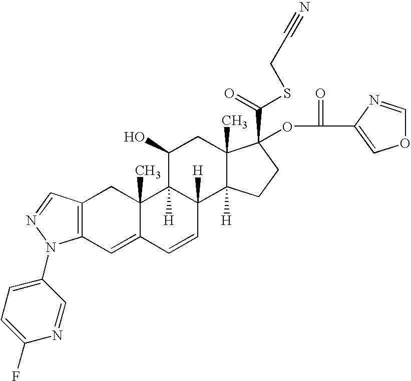 | 6 | (1R,3aS,3bS,10aR,10bS,11S,12aS)-1- {[cyanomethyl)sulfanyl]carbonyl}-7- (6-fluoropyridin-3-yl)-11-hydroxy- 10a,12a-dimethyl- 1,2,3,3a,3b,7,10,10a,10b,11,12,12a- dodecahydrocyclopenta[5,6]naphtho [1,2-f]indazol-1-yl 1,3-oxazole-4- carboxylate | 1H NMR (399.99 MHz, CdCl₃) δ 8.39 (1H, bs), 8.31 (1H, d, J = 0.91 Hz), 8.02 (1H, m), 7.96 (1H, d, J = 0.91 Hz), 7.55 (1H, s), 7.08 (1H, dd), 6.15-6.09 (2H, m), 5.89 (1H, d), 4.62 (1H, bs), 3.82 (1H, d, AB), 3.57 (1H, d, AB), 3.13 (1H, m), 3.05 (1H, d), 2.84 (1H, t), 2.74 (1H, d), 2.33-2.15 (2H, m), 2.12- 1.91 (3H, m), 1.74-1.63 (1H, m), 1.60 (1H, d), 1.26 (3H + 1H, s + bs), 1.13 (3H, s) | 616.2 |
| 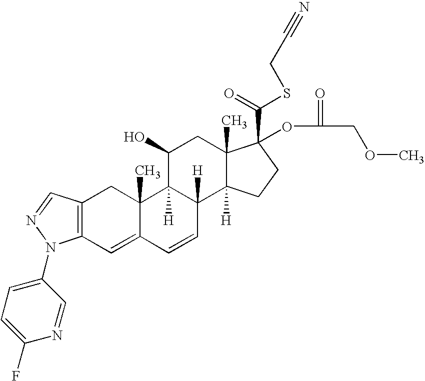 | 7 | (1R,3aS,3bS,10aR,10bS,11S,12aS)-1- {[(cyanomethyl)sulfanyl]carbonyl}-7- (6-fluoropyridin-3-yl)-11-hydroxy- 10a,12a-dimethyl- 1,2,3,3a,3b,7,10,10a,10b,11,12,12a- dodecahydrocyclopenta[5,6]naphtho [1,2-f]indazol-1-yl methoxyacetate | 1H NMR (399.99 MHz, CdCl₃) δ 8.38 (1H, s), 8.01 (1H, m), 7.54 (1H, s), 7.09 (1H, dd), 6.13 (1H, d), 6.12 (1H, s), 5.88 (1H, d), 4.56 (1H, bs), 4.06 (2H, s), 3.81 (1H, d, AB), 3.58 (1H, d, AB), 3.46 (3H, s), 3.12-2.99 (2H, m), 2.80 (1H, t), 2.69 (1H, d), 2.16- 1.93 (4H, m), 1.90-1.78 (1H, m), 1.71-1.58 (1H, m), 1.51 (1H, d), 1.24 (3H, s), 1.23 (1H, bs), 1.08 (3H, s) | 593.2 |
| 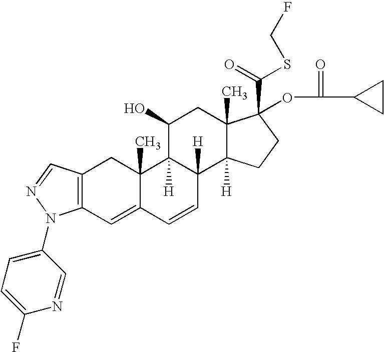 | 8 | (1R,3aS,3bS,10aR,10bS,11S,12aS)-1- {[(fluoromethyl)sulfanyl]carbonyl}-7- (6-fluoropyridin-3-yl)-11-hydroxy- 10a,12a-dimethyl- 1,2,3,3a,3b,7,10,10a,10b,11,12,12a- dodecahydrocyclopenta[5,6]naphtho [1,2-f]indazol-1-yl cyclopropanecarboxylate | 1H NMR (399.99 MHz, CdCl₃) δ 8.39 (1H, s), 8.01 (1H, m), 7.54 (1H, s), 7.09 (1H, dd), 6.13 (1H, d), 6.12 (1H, s), 6.07-5.62 (3H, m), 4.57 (1H, bs), 3.08- 2.98 (2H, m), 2.79 (1H, t), 2.71 (1H, d), 2.18 (1H, dd), 2.08-1.95 (3H, m), 1.94-1.83 (1H, m), 1.71-1.58 (2H, m), 1.52 (1H, d), 1.25 (3H, s), 1.17 (1H, bs), 1.10-0.98 (3H + 2H, s + m). 0.97-0.88 (2H, m) | 582.2 |
| 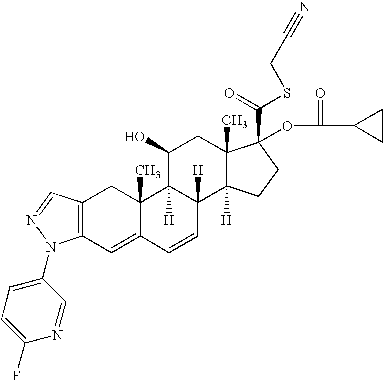 | 9 | (1R,3aS,3bS,10aR,10bS,11S,12aS)-1- {[(cyanomethyl)sulfanyl]carbonyl}-7- (6-fluoropyridin-3-yl)-11-hydroxy- 10a,12a-dimethyl-1,2,3,3a,3b,7,- 10,10a,10b,11,12,12a-dodecahydro- cyclopenta[5,6]naphtho[1,2-f]indazol- 1-yl cyclopropanecarboxylate | 1H NMR (399.99 MHz, CdCl₃) δ 8.39 (1H, s), 8.02 (1H, m), 7.54 (1H, s), 7.09 (1H, dd), 6.13 (1H, d), 6.12 (1H, s), 5.90 (1H, d), 4.57 (1H, bs), 3.79 (1H, d, AB), 3.56 (1H, d, AB), 3.09-2.93 (2H, m), 2.80 (1H, t), 2.71 (1H, d), 2.16 (1H, dd), 2.08-1.95 (3H, m), 1.93-1.83 (1H, m), 1.70-1.57 (2H, m), 1.52 (1H, d), 1.25 (3H, s), 1.22 (1H, bs), 1.06 (3H, s). 1.06-1.00 (2H, m), 0.97-0.89 (2H, m) | 589.2 |
| 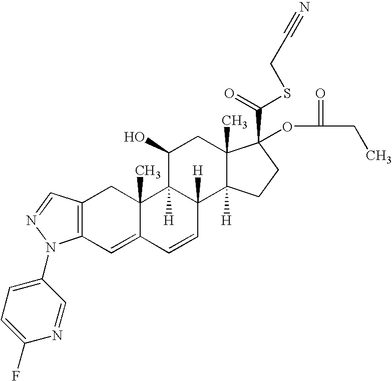 | 10 | (1R,3aS,3bS,10aR,10bS,11S,12aS)-1- {[(cyanomethyl)sulfanyl]carbonyl}-7- (6-fluoropyridin-3-yl)-11-hydroxy- 10a,12a-dimethyl- 1,2,3,3a,3b,7,10,10a,10b,11,12,12a- dodecahydrocyclopenta[5,6]naphtho [1,2-f]indazol-1-yl propanoate | 1H NMR (399.99 MHz, CdCl₃) δ 8.39 (1H, s), 8.01 (1H, m), 7.54 (1H, s), 7.08 (1H, dd), 6.13 (1H, d), 6.12 (1H, s), 5.89 (1H, d), 4.57 (1H, bs), 3.80 (1H, d, AB), 3.57 (1H, d, AB), 3.09-2.97 (2H, m), 2.80 (1H, t), 2.70 (1H, d), 2.38 (2H, q), 2.14 (1H, d), 2.08-1.93 (3H, m), 1.90-1.78 (1H, m), 1.68-1.58 (1H, m), 1.51 (1H, d), 1.25 (3H, s), 1.21 (1H, bs), 1.16 (3H, t), 1.06 (3H, s) | 577.2 |
| 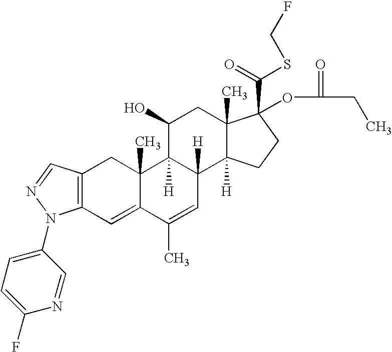 | 11 | (1R,3aS,3bS,10aR,10bS,11S,12aS)-1- {[(fluoromethyl)sulfanyl]carbonyl}-7- (6-fluoropyridin-3-yl)-11-hydroxy- 5,10a,12a-trimethyl- 1,2,3,3a,3b,7,10,10a,10b,11,12,12a- dodecahydrocyclopenta[5,6]naphtho [1,2-f]indazol-1-yl propanoate | 1H NMR (399.99 MHz, CdCl₃) δ 8.39 (1H, s), 8.03 (1H, m), 7.54 (1H, s), 7.10 (1H, dd), 6.25 (1H, s), 6.08-5.62 (3H, m), 5.77 (1H, s), 4.55 (1H, bs), 3.11- 2.99 (2H, m), 2.80-2.66 (2H, m), 2.37 (2H, dq), 2.16 (1H, d), 2.09-1.95 (2H, m), 1.88 (3H, s), 1.88-1.78 (1H, m), 1.66-1.56 (1H, m), 1.50 (1H, d), 1.21 (3H, s), 1.19-1.12 (4H, m), 1.04 (3H, s) | 584.2 |
| 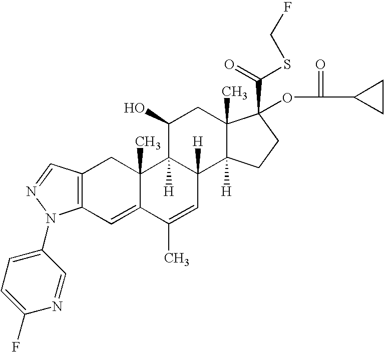 | 12 | (1R,3aS,3bS,10aR,10bS,11S,12aS)-1- {[(fluoromethyl)sulfanyl]carbonyl}-7- (6-fluoropyridin-3-yl)-11-hydroxy- 5,10a,12a-trimethyl-1,2,3,3a,3b,7,- 10,10a,10b,11,12,12a-dodecahydro- cyclopenta[5,6]naphtho[1,2-f]indazol- 1-yl cyclopropanecarboxylate | 1H NMR (399.99 MHz, CdCl₃) δ 8.39 (1H, s), 8.03 (1H, m), 7.54 (1H, s), 7.10 (1H, dd), 6.25 (1H, s), 6.07-5.61 (3H, m), 4.56 (1H, bs), 3.10-2.97 (2H, m), 2.80-2.66 (2H, m), 2.18 (1H, d), 2.08-1.96 (3H, m), 1.93-1.81 (4H, m), 1.69-1.61 (1H, m), 1.61-1.55 (1H, m), 1.51 (1H, d), 1.22 (3H, s), 1.19 (1H, bs), 1.08-1.00 (5H, m), 0.95-0.88 (2H, m) | 596.2 |
| 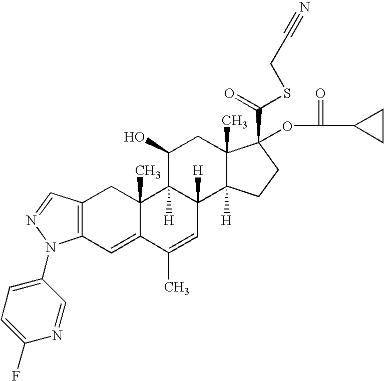 | 13 | (1R,3aS,3bS,10aR,10bS,11S,12aS)-1- {[cyanomethyl)sulfanyl]carbonyl}-7- (6-fluoropyridin-3-yl)-11-hydroxy- 5,10a,12a-trimethyl- 1,2,3,3a,3b,7,10,10a,10b,11,12,12a- dodecahydrocyclopenta[5,6]naphtho [1,2-f]indazol-1-yl cyclopropanecarboxylate | 1H NMR (399.99 MHz, CdCl₃) δ 8.39 (1H, s), 8.03 (1H, m), 7.55 (1H, s), 7.10 (1H, dd), 6.25 (1H, s), 5.79 (1H, s), 4.57 (1H, bs), 3.79 (1H, d, AB), 3.57 (1H, d, AB), 3.09-2.94 (2H, m), 2.82-2.66 (2H, m), 2.16 (1H, d), 2.08-1.96 (3H, m), 1.93-1.81 (4H, m), 1.69-1.57 (2H, m), 1.50 (1H, d), 1.28-1.20 (4H, s), 1.11-1.00 (5H, m), 0.98-0.88 (2H, m) | 603.2 |
| 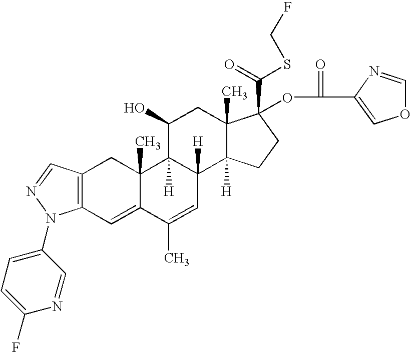 | 14 | (1R,3aS,3bS,10aR,10bS,11S,12aS)-1- {[(fluoromethyl)sulfanyl]carbonyl}-7- (6-fluoropyridin-3-yl)-11-hydroxy- 5,10a,12a-trimethyl- 1,2,3,3a,3b,7,10,10a,10b,11,12,12a- dodecahydrocyclopenta[5,6]naphtho [1,2-f]indazol-1-yl 1,3-oxazole-4- carboxylate | 1H NMR (399.99 MHz, CdCl₃) δ 8.39 (1H, s), 8.30 (1H, s), 8.03 (1H, m), 7.95 (1H, s), 7.55 (1H, s), 7.10 (1H, dd), 6.25 (1H, s), 6.07-5.62 (3H, m), 4.61 (1H, bs), 3.15 (1H, m), 3.05 (1H, d), 2.83-2.70 (2H, m), 2.31 (1H, d), 2.25-2.14 (1H, m), 2.14-1.91 (3H, m), 1.86 (3H, s), 1.71-1.55 (2H, m), 1.23 (3H, s), 1.20 (1H, bs), 1.10 (3H, s) | 623.2 |
| 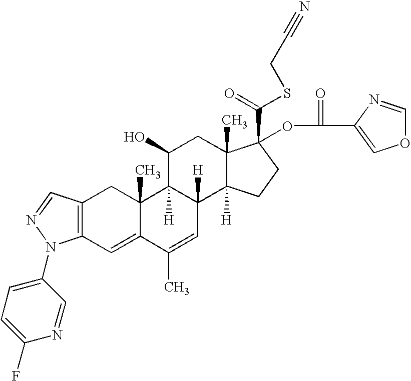 | 15 | (1R,3aS,3bS,10aR,10bS,11S,12aS)-1- {[cyanomethyl)sulfanyl]carbonyl}-7- (6-fluoropyridin-3-yl)-11-hydroxy- 5,10a,12a-trimethyl- 1,2,3,3a,3b,7,10,10a,10b,11,12,12a- dodecahydrocyclopenta[5,6]naphtho [1,2-f]indazol-1-yl 1,3-oxazole-4- carboxylate | 1H NMR (399.99 MHz, CdCl₃) δ 8.39 (1H, s), 8.31 (1H, s), 8.03 (1H, m), 7.96 (1H, s), 7.56 (1H, s), 7.10 (1H, dd), 6.25 (1H, s), 5.77 (1H, m), 4.62 (1H, bs), 3.81 (1H, d, AB), 3.57 (1H, d, AB), 3.20-3.00 (2H, m), 2.84-2.69 (2H, m), 2.33-2.15 (2H, m), 2.11-2.01 (2H, m), 2.01-1.90 (1H, m), 1.86 (3H, s), 1.74-1.61 (1H, m), 1.57-1.46 (1H, m), 1.26 (1H, bs), 1.23 (3H, s), 1.11 (3H, s) | 630.2 |
| 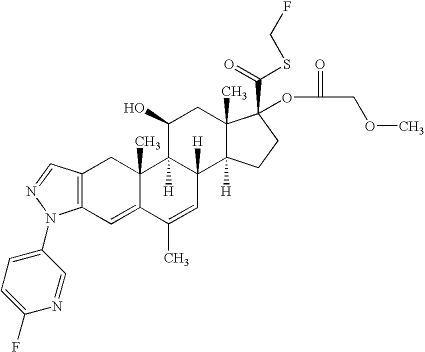 | 16 | (1R,3aS,3bS,10aR,11S,12aS)-1- {[(fluoromethyl)sulfanyl]carbonyl}-7- (6-fluoropyridin-3-yl)-11-hydroxy- 5,10a,12a-trimethyl- 1,2,3,3a,3b,7,10,10a,10b,11,12,12a- dodecahydrocyclopenta[5,6]naphtho [1,2-f]indazol-1-yl methoxyacetate | 1H NMR (399.99 MHz, CdCl₃) δ 8.39 (1H, s), 8.03 (1H, m), 7.54 (1H, s), 7.10 (1H, dd), 6.25 (1H, s), 6.06-5.63 (3H, m), 4.55 (1H, bs), 4.06 (2H, s), 3.45 (3H, s), 3.20-2.99 (2H, m), 2.80-2.65 (2H, m), 2.17- 1.93 (4H, m), 1.87 (3H, s), 1.86-1.76 (1H, m), 1.69- 1.56 (1H, m), 1.50 (1H, d), 1.34 (1H, t), 1.21 (3H, s), 1.17 (1H, bs), 1.06 (3H, s) | 600.2 |
| 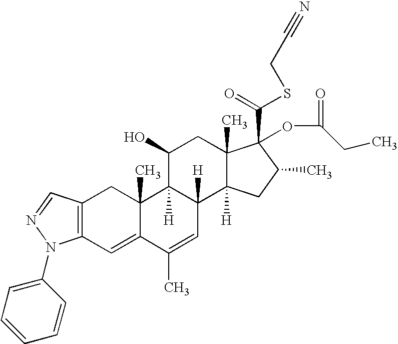 | 17 | (1R,2R,3aS,3bS,10aR,10bS,11S,12aS)- 1-{[(cyanomethyl)sulfanyl]carbonyl}- 11-hydroxy-2,5,10a,12a-tetramethyl-7- phenyl-1,2,3,3a,3b,7,10,10a,- 10b,11,12,12a-dodecahydro- cyclopenta[5,6]naphtho[1,2-f]indazol- 1-yl propanoate | 1H NMR (400.0 MHz, CdCl₃) δ 7.45-7.57 (m, 5H), 7.32-7.40 (m, 1H), 6.35 (s, 1H), 5.69 (s, 1H), 4.54 (s, 1H), 3.72 (s, 2H), 3.32-3.41 (m, 1H), 3.02 (d, 1H), 2.64-2.74 (m, 2H), 2.38 (q, 2H), 1.90- 2.12 (m, 4H), 1.88 (s, 3H), 1.57-1.61 (m, 1H), 1.47 (dd, 1H), 1.21 (s, 3H), 1.12-1.19 (m, 7H), 1.03 (d, 3H) | 586.3 |
| 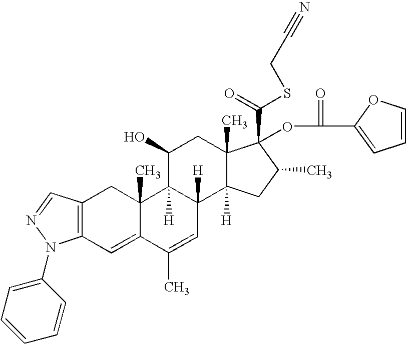 | 18 | (1R,2R,3aS,3bS,10aR,10bS,11S,12aS)- 1-{[(cyanomethyl)sulfanyl]carbonyl}- 11-hydroxy-2,5,10a,12a-tetramethyl-7- phenyl-1,2,3,3a,3b,7,10,10a,- 10b,11,12,12a-dodecahydro- cyclopenta[5,6]naphtho[1,2-f]indazol- 1-yl furan-2-carboxylate | 1H NMR (400.0 MHz, CdCl₃) δ 1.10 (d, 3H), 1.20- 1.24 (m, 7H), 1.53 (d, 1H), 1.63 (dd, 1H), 1.89 (s, 3H), 1.98-2.13 (m, 3H), 2.21 (dd, 1H), 2.69-2.79 (m, 2H), 3.05 (d, 1H), 3.41-3.52 (m, 1H), 3.70- 3.80 (m, 2H), 4.56-4.62 (m, 1H), 5.71 (s, 1H), 6.37 (s, 1H), 6.51-6.55 (m, 1H), 7.20 (d, 1H), 7.34- 7.40 (m, 1H), 7.47-7.57 (m, 5H), 7.60-7.62 (m, 1H). | 624.5 |

### Human Glucocorticoid Receptor (GR) Assay

The assay is based on a commercial kit from Panvera/Invitrogen (Part number P2893). The assay technology is fluorescence polarization. The kit utilises recombinant human GR (Panvera, Part number P2812), a Fluoromone™ labelled tracer (GS Red, Panvera, Part number P2894) and a Stabilizing Peptide 10× (Panvera, Part number P2815). The GR and Stabilizing Peptide reagents are stored at −70° C. while the GS Red is stored at −20° C. Also included in the kit are 1M DTT (Panvera, Part number P2325, stored at −20° C.) and GR Screening buffer 10× (Panvera, Part number P2814, stored at −70° C. initially but once thawed stored at room temperature). Avoid repeated freeze/thaws for all reagents. The GR Screening buffer 10× comprises 100 mM potassium phosphate, 200 mM sodium molybdate, 1 mM EDTA and 20% DMSO.

Test compounds (1 μL) and controls (1 μL) in 100% DMSO were added to black polystyrene 384-well plates (Greiner low volume black flat-bottom, part number 784076). 0% control was 100% DMSO and 100% control was 10 μM Dexamethasone. Background solution (8 μL; assay buffer 10×, Stabilizing Peptide, DTT and ice cold MQ water) was added to the background wells. GS Red solution (7 μL; assay buffer 10×, Stabilizing Peptide, DTT, GS Red and ice cold water) was added to all wells except background wells. GR solution (7 μL; assay buffer 10×, Stabilizing Peptide, DTT, GR and ice cold water) was added to all wells. The plate was sealed and incubated in a dark at room temperature for 2 hours. The plate was read in an Analyst plate reader (LJL Biosystems/Molecular Devices Corporation) or other similar plate reader capable of recording fluorescence polarization (excitation wavelength 530 nm, emission wavelength 590 nm and a dichroic mirror at 561 nm). The IC₅₀ values were calculated using XLfit model 205 and are shown in Table 1.

TABLE 1

| | Inhibition of GR | | Inhibition of GR |
|---|---|---|---|
| Example No. | binding, IC₅₀ (nM) | Example No. | binding, IC₅₀ (nM) |
| 1 | 2.3 | 2 | 2.1 |
| 3 | 1.4 | 4 | 0.8 |
| 5 | 1.5 | 6 | 1.8 |
| 7 | 0.87 | 8 | 1.4 |
| 9 | 1 | 10 | 0.83 |
| 11 | 3.6 | 12 | 1.6 |
| 13 | 1.5 | 14 | 2.9 |
| 15 | 3.7 | 16 | 1.5 |
| 17 | 4.8 | 18 | 12 |

1. A compound of formula 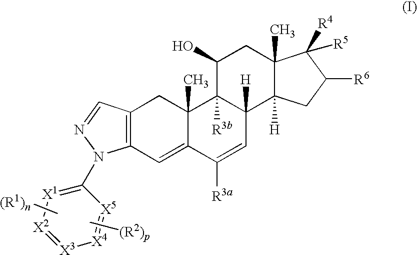
wherein
X¹, X², X³, X⁴ and X⁵ each independently represent CH or a nitrogen atom, provided that no more than two of X¹, X², X³, X⁴ and X⁵ may simultaneously represent a nitrogen atom;
n and p each independently represent 0 or 1;
R¹ represents a halogen atom or a methyl or a methoxy group;
R² represents a halogen atom, —C(O)OCH₃, —C(O)—S—CH₂CN, —C(O)—S—CH₃, —C(O)-heterocyclyl, —SO₂CH₃, a C₂-C₆ alkenyl group, or a methyl group optionally substituted by halogen, hydroxyl, methoxy, —OCH₂CH═CH₂ or —NR⁷R⁸;
R^{3a} represents a hydrogen atom or methyl group and R^{3b} represents a hydrogen or fluorine atom;
R⁴ represents —C(O)—S—C(O)N(CH₃)₂, —C(O)CH₂Cl, —C(O)—Y—CH(R¹¹)—R⁹ or —C(O)—CH(R¹¹)—Y—R⁹;
R⁵ represents —OCH₂SCH₃, —O—C(O)—R¹⁰, —O—C(O)—NH—R¹⁰, —O—C(O)—O—R¹⁰ or —O—C(O)—S—R¹⁰;
R⁶ represents a hydrogen or a halogen atom or a hydroxyl or methyl group;
R⁷ and R⁸ each independently represent a hydrogen atom, or a C₁-C₃ alkyl or a C₁-C₃ hydroxyalkyl group, or
R⁷ and R⁸ together with the nitrogen atom to which they are attached form a 3- to 8-membered saturated or partially saturated heterocyclic ring optionally containing a further ring heterogroup selected from nitrogen, S(O)ₘ and oxygen, the heterocyclic ring being optionally substituted by at least one substituent selected from hydroxyl, C₁-C₃ alkyl and C₁-C₃ hydroxyalkyl;
m is 0, 1 or 2;
Y represents an oxygen or sulphur atom or a group>NH;
R⁹ represents hydrogen, halogen, cyano, —S—CN, —C(O)N(R¹²)₂, C₁-C₆ alkoxycarbonyl, C₁-C₆ alkylcarbonyl (optionally substituted by —OC(O)CH₃), C₁-C₆ alkylcarbonyloxy, C₁-C₆ alkoxy, C₁-C₆ alkylthio, —C(O)—S—C₁-C₆ alkyl, —C(═CH₂)—O—CH₂OCH₃, C₁-C₆ alkyl, C₂-C₆ alkenyl, C₂-C₆ alkynyl or C₃-C₇ cycloalkyl, the latter four groups being optionally substituted by one or more substituents independently selected from halogen, hydroxyl, cyano, hydroxymethyl, C₁-C₄ alkoxy and C₁-C₄ alkylcarbonyloxy;
R¹⁰ represents C₁-C₆ alkyl (optionally substituted by halogen, C₁-C₄ alkoxy, C₁-C₄ alkylcarbonyloxy or C₃-C₇ cycloalkyl) or a 3- to 10-membered saturated or unsaturated carbocyclic or heterocyclic ring system which ring system may be optionally substituted by at least one substituent selected from halogen, carboxyl, hydroxyl, oxo, nitro, cyano, mercapto, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylthio, C₁-C₆ alkylsulphinyl, C₁-C₆ alkylsulphonyl, C₁-C₆ alkylcarbonyl, C₁-C₆ alkylcarbonyloxy, C₁-C₆ alkoxycarbonyl, amino, carboxamido, (mono)C₁-C₆ alkylamino, (di)C₁-C₆ alkylamino and phenyl;
R¹¹ represents a hydrogen atom or a methyl group; and
each R¹² independently represents a hydrogen atom or a methyl group;
or a pharmaceutically acceptable salt thereof.

2. A compound according to claim 1, wherein either X¹, X², X³, X⁴ and X⁵ each represent CH, or one of X¹, X², X³, X⁴ and X⁵ represents a nitrogen atom and the others represent CH.

3. A compound according to claim 1, wherein n is 0 or 1, p is 0 and R¹ represents a halogen atom.

4. A compound according to claim 1, wherein R^{3a} represents a hydrogen atom or methyl group and R^{3b} represents a hydrogen atom.

5. A compound according to claim 1, wherein R⁴ represents —C(O)—Y—CH(R¹¹)—R⁹ or —C(O)—CH(R¹¹)—Y—R⁹.

6. A compound according to claim 1, wherein Y represents an oxygen or sulphur atom.

7. A compound according to claim 1, wherein R⁹ represents hydrogen, halogen, cyano, —S—CN, —C(O)N(R¹²)₂, C₁-C₂ alkoxycarbonyl, C₁-C₂ alkylcarbonyl (optionally substituted by —OC(O)CH₃), C₁-C₂ alkylcarbonyloxy, C₁-C₂ alkoxy, C₁-C₂ alkylthio, —C(O)—S—C₁-C₂ alkyl, —C(═CH₂)—O—CH₂OCH₃, C₁-C₆ alkyl, C₂-C₄ alkenyl, C₂-C₄ alkynyl or C₃-C₆ cycloalkyl, the latter four groups being optionally substituted by one or more substituents independently selected from halogen, hydroxyl, cyano, hydroxymethyl, C₁-C₄ alkoxy and C₁-C₄ alkylcarbonyloxy.

8. A compound according to claim 1, wherein R⁹ represents hydrogen, halogen, cyano, methyl, hydroxymethyl or methylcarbonyl.

9. A compound according to claim 1, wherein R⁵ represents —O—C(O)—R¹⁰.

10. A compound according to claim 1, wherein R¹⁰ represents C₁-C₄ alkyl optionally substituted by C₁-C₂ alkoxy, or a cyclopropyl, oxazolyl or furanyl ring.

11. A compound according to claim 1 being:
(1R,3aS,3bS,10aR,10bS,11S,12aS)-1-{[(Cyanomethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl methoxyacetate,
(1R,3aS,3bS,10aR,10bS,11S,12aS)-1-{[(Cyanomethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl propanoate,
(1R,3aS,3bS,10aR,10bS,11S,12aS)-1-{[(Fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl propanoate, or
(1R,3aS,3bS,10aR,10bS,11S,12aS)-1-{[(Fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl methoxyacetate,
(1R,3aS,3bS,10aR,10bS,11S,12aS)-1-{[(fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl 1,3-oxazole-4-carboxylate,
(1R,3aS,3bS,10aR,10bS,11S,12aS)-1-{[(cyanomethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl 1,3-oxazole-4-carboxylate,
(1R,3aS,3bS,10aR,10bS,11S,12aS)-1-{[(cyanomethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl methoxyacetate,
(1R,3aS,3bS,10aR,10bS,11S,12aS)-1-{[(fluoromethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl cyclopropanecarboxylate,
(1R,3aS,3bS,10aR,10bS,11S,12aS)-1-{[(cyanomethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl cyclopropanecarboxylate,
(1R,3aS,3bS,10aR,10bS,11S,12aS)-1-{[(cyanomethyl)sulfanyl]carbonyl}-7-(6-fluoropyridin-3-yl)-11-hydroxy-10a,12a-dimethyl-1,2,3,3a,3b,7,10,10a,10b,11,12,12a-dodecahydrocyclopenta[5,6]naphtho[1,2-f]indazol-1-yl propanoate,
(1R,3aS,3bS,10aR,10bS,11S,12aS)-1-{[(fluoromethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl propanoate,
(1R,3aS,3bS,10aR,10bS,11S,12aS)-1-{[(fluoromethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl cyclopropanecarboxylate,
(1R,3aS,3bS,10aR,10bS,11S,12aS)-1-{[(cyanomethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl cyclopropanecarboxylate,
(1R,3aS,3bS,10aR,10bS,11S,12aS)-1-{[(fluoromethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl 1,3-oxazole-4-carboxylate,
(1R,3aS,3bS,10aR,10bS,11S,12aS)-1-{[(cyanomethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl 1,3-oxazole-4-carboxylate,
(1R,3aS,3bS,10aR,11S,12aS)-1-{[(fluoromethyl)sulfanyl]carbonyl}-7-(6-fluoropyridin-3-yl)-11-hydroxy-5,10a,12a-trimethyl-1,2,3,3a,3b,7,10,10a,10b,11,12,12a-dodecahydrocyclopenta[5,6]naphtho[1,2-f]indazol-1-yl methoxyacetate,
(1R,2R,3aS,3bS,10aR,10bS,11S,12aS)-1-{[(cyanomethyl)sulfanyl]carbonyl}-11-hydroxy-2,5,10a,12a-tetramethyl-7-phenyl-1,2,3,3a,3b,7,10,10a,10b,11,12,12a-dodecahydrocyclopenta[5,6]naphtho[1,2-f]indazol-1-yl propanoate,
(1R,2R,3aS,3bS,10aR,10bS,11S,12aS)-1-{[(cyanomethyl)sulfanyl]carbonyl}-11-hydroxy-2,5,10a,12a-tetramethyl-7-phenyl-1,2,3,3a,3b,7,10,10a,10b,11,12,12a-dodecahydrocyclopenta[5,6]naphtho[1,2-f]indazol-1-yl furan-2-carboxylate,
or a pharmaceutically acceptable salt thereof.

12. A process for the preparation of a compound of formula (I) or a pharmaceutically acceptable salt thereof as defined in claim 1 which comprises
(i) reacting a compound of formula (II) 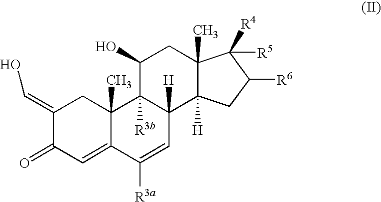
wherein R^{3a}, R^{3b}, R⁴, R⁵ and R⁶ are as defined in formula (I), with a compound of formula (III) or an acid addition salt thereof 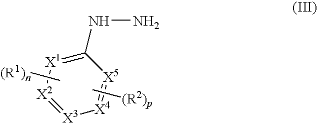
wherein n, p, R¹, R², X¹, X², X³, X⁴ and X⁵ are as defined in formula (I); or
(ii) when R⁴ represents —C(O)—Y—CH(R¹¹)—R⁹ and Y represents a sulphur atom, reacting a compound of formula (IV) 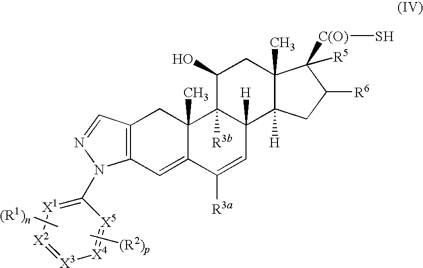
where n, p, X¹, X², X³, X⁴, X⁵, R¹, R², R^{3a}, R^{3b}, R⁵ and R⁶ are as defined in formula (I), with a compound of formula (V), R⁹—CH(R¹¹)-L, where L represents a leaving group and R⁹ and R¹¹ are as defined in formula (I);
and optionally thereafter carrying out one or more of the following procedures:
converting a compound of formula (I) into another compound of formula (I)
removing any protecting groups
forming a pharmaceutically acceptable salt.

13. A pharmaceutical composition comprising a compound of formula (I) as claimed in claim 1 or a pharmaceutically acceptable salt thereof in association with a pharmaceutically acceptable adjuvant, diluent or carrier.

14. A method of treating asthma, chronic obstructive pulmonary disease or allergic rhinitis which comprises administering to the patient a therapeutically effective amount of a compound of formula (I), or a pharmaceutically acceptable salt thereof, as defined in claim 1.
